SEQUENCE LISTING (11 sequences)

SEQ ID NO: 1 - protein, 124 aa, Artificial Sequence
  source: /note=“Description of Artificial Sequence Synthetic polypeptide”
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 2 - protein, 108 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, Artificial Sequence
  source: /note=“Description of Artificial Sequence Synthetic peptide”
SYGMH

SEQ ID NO: 4 - protein, 17 aa, Artificial Sequence
  source: /note=“Description of Artificial Sequence Synthetic peptide”
VIWFDGTKKYYTDSVKG

SEQ ID NO: 5 - protein, 15 aa, Artificial Sequence
  source: /note=“Description of Artificial Sequence Synthetic peptide”
DRGIGARRGPYYMDV

SEQ ID NO: 6 - protein, 11 aa, Artificial Sequence
  source: /note=“Description of Artificial Sequence Synthetic peptide”
RASQSISSYLN

SEQ ID NO: 7 - protein, 7 aa, Artificial Sequence
  source: /note=“Description of Artificial Sequence Synthetic peptide”
AASSLQS

SEQ ID NO: 8 - protein, 9 aa, Artificial Sequence
  source: /note=“Description of Artificial Sequence Synthetic peptide”
QQSYSTPLT

SEQ ID NO: 9 - protein, 10 aa, Artificial Sequence
  source: /note=“Description of Artificial Sequence Synthetic peptide”
GFAFSSYGMH

SEQ ID NO: 10 - protein, 453 aa, Artificial Sequence
  source: /note=“Description of Artificial Sequence Synthetic polypeptide”
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0308259 A1
(43) Pub. Date: Oct. 1, 2020

(54) METHODS FOR TREATING ALZHEIMER'S DISEASE

(71) Applicant: Biogen International Neuroscience GmbH, Baar (CH)

(72) Inventors: Brendon Phillip Boot, Somerville, MA (US); Jeffrey Joseph Sevigny, Binningen (CH); Leslie Lugene Williams, Sherborn, MA (US)

(21) Appl. No.: 16/307,364

(22) PCT Filed: Jun. 6, 2017
(86) PCT No.: PCT/EP2017/063711

§ 371 Date: Dec. 5, 2018

Related U.S. Application Data
(60) Provisional application No. 62/435,531, filed on Dec. 16, 2016.
(60) Provisional application No. 62/346,818, filed on Jun. 7, 2016.

(51) Int. Cl. C07K 16/18; A61P 25/28

(52) U.S. Cl. CPC C07K 16/18; A61P 25/28; C07K 2317/515; C07K 2317/51; C07K 2317/52

(57) ABSTRACT

Provided are methods for treating Alzheimer's disease in a human subject in need thereof when the subject develops an Amyloid Related Imaging Abnormality (ARIA) during a treatment regimen comprising administration of multiple doses of an anti-beta-amyloid antibody (e.g., BIIB037) to the subject.

### CROSS-REFERENCE TO RELATED APPLICATIONS

This application claims the benefit of priority of U.S. Provisional Application Nos. 62/346,818, filed Jun. 7, 2016, and 62/435,531 filed Dec. 16, 2016, the content of both of which are incorporated by reference herein in their entireties.

### TECHNICAL FIELD

This disclosure relates generally to methods for treating Alzheimer's disease.

### BACKGROUND

Alzheimer's disease (AD) is a progressive neurodegenerative disorder clinically characterized by cognitive impairment, behavioral disturbances, psychiatric symptoms, and disability in activities of daily living. These clinical manifestations constitute AD dementia.

AD International estimates that the number of people living with dementia worldwide will increase from the current value of 35.6 million to 115.4 million by 2050. Being the most common cause of dementia, AD accounts for 60 to 80% of dementia cases. In the United States, it is estimated that 5.3 million Americans suffer from dementia caused by AD, and that by 2050 the prevalence will double or triple unless an effective treatment is found.

Clinical research criteria for dementia due to AD have been recently updated and conforming to the current concept of the disease, a diagnostic framework was developed to embrace pre-dementia stages of AD (e.g., prodromal AD). The main neuropathological hallmarks of the disease are (i) extracellular senile (neuritic) plaques containing aggregated β-amyloid (Aβ) peptides and (ii) intraneuronal neurofibrillary tangles (NFTs) composed of abnormal hyperphosphorylated Tau protein. Although the pathogenesis of these plaques and tangles and how they contribute to the clinical syndrome remain to be fully elucidated, the leading hypothesis the “amyloid cascade” proposes that the driving force behind the disease process is the accumulation of Aβ resulting from an imbalance between Aβ production and Aβ clearance in the brain.

Aβ is a peptide generated from the metabolism of amyloid precursor protein. Several Aβ peptide alloforms exist (e.g., Aβ40, Aβ42). These monomeric peptides have a variable tendency to aggregate into higher order dimers and oligomers. Through a process of fibrillogenesis, soluble oligomers may transition into insoluble deposits having a 13 pleated sheet structure. These deposits are also referred to as amyloid plaques and hence are composed of predominantly fibrillar amyloid. Both soluble and fibrillar forms of Aβ appear to contribute to the disease process.

Biomarker, clinicopathologic, and cohort studies suggest that the disease process commences 10 to 20 years before the clinical onset of symptoms, and some of the early pathological findings include the deposition of neocortical neuritic plaques and mesial temporal NFTs followed years later by neocortical NFTs.

There are currently no therapies that modify the course of Alzheimer's disease. Currently approved therapies provide only modest symptomatic benefit and do not attenuate the course of the disease. Several potential disease-modifying drug candidates are currently under investigation. These candidates include small molecules and immunotherapy (active and passive) that target the Aβ pathway and aim to provide therapeutic benefit by reducing either soluble or insoluble forms of Aβ in the brain and cerebrospinal fluid (CSF).

In response to guidance issued by the U.S. Food and Drug Administration (FDA) to various sponsors on the conduct of clinical trials of amyloid-modifying agents for the treatment of AD, the Alzheimer's Association Research Roundtable convened a Workgroup in July 2010. The Workgroup was composed of academic and industry representatives identified on the basis of their expertise and interest in this area. It was tasked with the objective of providing expert advice regarding the FDA's concerns related to magnetic resonance imaging (MRI) abnormalities, including signal changes thought to represent vasogenic edema (VE) and microhemorrhages (mH). MRI signal changes were first observed in trials of a monoclonal antibody against β-amyloid, and have since been associated with other amyloid-modifying therapies.

While the exact pathophysiologic mechanisms of these MRI abnormalities have not been determined, VE and mH are typically detected on different MRI sequences. They appear to represent a spectrum of image abnormalities which may share some common underlying pathophysiological mechanism, both in the natural history of AD and in the setting of amyloid-modifying therapeutic approaches. The Workgroup suggested referring to this spectrum as Amyloid Related Imaging Abnormalities (ARIA).

Despite the likelihood of shared underlying mechanisms, there may be instances in which it is useful to describe specific phenomena. Thus, the Workgroup further refined the terminology: ARIA-E refers to the MR signal alterations thought to represent VE and related extravasated fluid phenomena. ARIA-H refers to the MR signal alterations attributable to mH and hemosiderosis.

ARIA-E most commonly manifests as increased MR signal intensity on FLAIR or other T2-weighted sequences in the parenchyma and/or leptomeninges in the parietal, occipital, and frontal lobes, but has also been observed in the cerebellum and brainstem. The presence of Apolipoprotein E ε4 allele, ApoE ε4, has been found to be a significant risk factor for the development of ARIA-E.

There are currently very limited publicly available data regarding the clinical course associated with ARIA-E occurring in the setting of clinical trials of amyloid modifying therapies. The Workgroup reviewed the data from bapineuzumab trials, but it noted that it was unknown whether ARIA seen in other amyloid-modifying therapies will have similar clinical course. In any event, the pathophysiological mechanisms underlying vasogenic edema remain to be elucidated.

mH are generally attributed to one of two etiologies: small vessel angiopathy and cerebral amyloid angiopathy (CAA). The prevalence of mH is significantly increased in elderly individuals with cardiovascular risk factors and/or evidence of a previous cerebrovascular event. In AD, mH and superficial siderosis are attributed to leakage of blood from CAA vessels. CAA is believed to weaken the vessel wall, increasing the risk of micro leaks of blood into adjacent brain, forming mH. Moreover, there are limited publicly available data on incident mH in the setting of ARIA-E associated with amyloid-modifying therapies.

Preliminary reports of ARIA occurrence in therapeutic strategies aimed at decreasing production of specific Aβ peptides suggest that decreasing Aβ1-42 or altering the ratio of various Aβ species might change the dynamics of amyloid production and clearance, resulting in ARIA. It is possible that direct removal of amyloid from the vessel wall would be associated with compromise in the vascular integrity. Alternatively, there may be amyloid related endothelial cell dysfunction resulting in increased vascular permeability, which might explain the similarity to increased permeability. It is also possible that there is a focal inflammatory component that would result in both ARIA-E and ARIA-H, as suggested by the pathology reports from patients with CAA. Normal CSF has also been reported in inflammatory CAA, and it is possible that focal amyloid-related vascular inflammation may play a role in some cases of ARIA. It also remains unknown whether different forms of immunotherapy or specific antibodies are more or less likely to be associated with ARIA.

The incidence of ARIA in patients undergoing treatment for Alzheimer's disease continues to be a persistent problem. While there are a number of potential mechanisms of action to target, solutions to the problem have not been found.

Thus, there is a need in the art for methods to reduce the incidence of ARIA in susceptible Alzheimer's disease patients during AD treatment protocols.

### SUMMARY

This disclosure fulfills the need in the art for methods to reduce the incidence of ARIA in Alzheimer's disease patients during Alzheimer's disease (AD) treatment protocols.

In one aspect, the disclosure features a method for treating AD in a human subject in need thereof. The method involves administering to the human subject multiple doses of an anti-beta-amyloid antibody, wherein the subject develops an Amyloid Related Imaging Abnormality (ARIA) during treatment with the anti-beta-amyloid antibody. The ARIA can be, for example: (i) ARIA-E that is moderate or severe and is accompanied by no clinical symptoms; (ii) ARIA-E that is mild, moderate, or severe and is accompanied by mild, moderate, or severe clinical symptoms, or clinical symptoms meeting the “other medically important” serious criteria; (iii) ARIA-H with 5 to 9 cumulative microhemorrhages and accompanied by no clinical symptoms; (iv) ARIA-H with 1 to 9 cumulative microhemorrhages and accompanied by mild, moderate, or severe clinical symptoms, or clinical symptoms meeting the “other medically important” serious criteria; (v) ARIA-H with 2 cumulative areas of superficial siderosis and accompanied by no clinical symptoms; or (vi) ARIA-H with 1 or 2 cumulative areas of superficial siderosis and accompanied by mild, moderate, or severe clinical symptoms, or clinical symptoms meeting the “other medically important” serious criteria. After the onset of ARIA in the subject, administration of the anti-beta-amyloid antibody to the subject is suspended until the ARIA resolves (and if there are clinical symptoms, until they also resolve). The method further involves resuming administration to the subject of the same dose of the anti-beta-amyloid antibody that was administered immediately prior to the subject developing the ARIA.

In some embodiments, the multiple doses of the anti-beta-amyloid antibody are doses of the same amount. In certain instances, the multiple doses are each 1 mg/kg of body weight of the subject. In certain instances, the multiple doses are each 3 mg/kg of body weight of the subject. In certain instances, the multiple doses are each 6 mg/kg of body weight of the subject. In certain instances, the multiple doses are each 10 mg/kg of body weight of the subject. In certain instances, the multiple doses are each 12 mg/kg of body weight of the subject. In certain instances, the multiple doses are each 15 mg/kg of body weight of the subject. In certain instances, the multiple doses are each 18 mg/kg of body weight of the subject. In certain instances, the multiple doses are each 20 mg/kg of body weight of the subject. In certain instances, the multiple doses are each 24 mg/kg of body weight of the subject. In certain instances, the multiple doses are each 30 mg/kg of body weight of the subject.

In other embodiments, the multiple doses of the anti-beta-amyloid antibody comprise doses of different amounts. In certain instances, the multiple doses comprise 1 mg/kg and 3 mg/kg of body weight of the subject. In certain instances, the multiple doses comprise 1 mg/kg, 3 mg/kg, and 6 mg/kg of body weight of the subject. In certain instances, the multiple doses comprise 3 mg/kg and 6 mg/kg of body weight of the subject. In certain instances, the multiple doses comprise 1 mg/kg, 3 mg/kg, 6 mg/kg, and 10 mg/kg of body weight of the subject. In certain instances, the multiple doses comprise 3 mg/kg, 6 mg/kg, and 10 mg/kg of body weight of the subject. In certain instances, the multiple doses comprise 3 mg/kg, 6 mg/kg, 10 mg/kg and 12 mg/kg of body weight of the subject. In certain instances, the multiple doses comprise 3 mg/kg, 6 mg/kg, 10 mg/kg and 15 mg/kg of body weight of the subject.

In some embodiments, wherein the subject is an ApoE4 carrier, the multiple doses comprise two or more of the doses 1 mg/kg, 3 mg/kg, 6 mg/kg, or 10 mg/kg of body weight of the subject. In some embodiments, wherein the subject is an ApoE4 non-carrier, the multiple doses comprise two or more of the doses 1 mg/kg, 3 mg/kg, 6 mg/kg, 10 mg/kg, 15 mg/kg, or 30 mg/kg of body weight of the subject.

In certain embodiments, the method further involves subsequently administering the anti-beta-amyloid antibody at a dose that is higher than the dose that is administered upon resumption of administration after resolution of the ARIA.

In some embodiments, the multiple doses are administered at intervals of 4 weeks.

In some embodiments, the number of multiple doses administered to the subject prior to the onset of the ARIA is 2 to 14 (e.g., 2, 3, 4, 5, 6, 7, 8, 9, 10, 11, 12, 13, or 14 doses). In other embodiments, the number of multiple doses administered to the subject prior to the onset of the ARIA is 2 to 5. In one embodiment, the number of multiple doses administered to the subject prior to the onset of the ARIA is 2. In one embodiment, the number of multiple doses administered to the subject prior to the onset of the ARIA is 3. In one embodiment, the number of multiple doses administered to the subject prior to the onset of the ARIA is 4. In one embodiment, the number of multiple doses administered to the subject prior to the onset of the ARIA is 5.

In certain embodiments, the administering to the human subject multiple doses of the anti-beta-amyloid antibody comprises, in order, beginning with step (a) performing two or more of the following administering steps prior to the onset of the ARIA:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 3 mg/kg of body weight of the subject;

(f) 4 weeks after step (e), administering the antibody to the subject in an amount of 3 mg/kg of body weight of the subject;

(g) 4 weeks after step (f), administering the antibody to the subject in an amount of 6 mg/kg of body weight of the subject; and

(h) in consecutive intervals of 4 weeks after step (g), administering the antibody to the subject in an amount of 6 mg/kg of body weight of the subject.

In certain embodiments, the method involves, after resolution of the ARIA (and resolution of any clinical symptoms), performing from the following administering steps, in order, those steps that were not performed prior to the onset of the ARIA:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 3 mg/kg of body weight of the subject;

(f) 4 weeks after step (e), administering the antibody to the subject in an amount of 3 mg/kg of body weight of the subject;

(g) 4 weeks after step (f), administering the antibody to the subject in an amount of 6 mg/kg of body weight of the subject; and

(h) in consecutive intervals of 4 weeks after step (g), administering the antibody to the subject in an amount of 6 mg/kg of body weight of the subject.

In certain embodiments, the method involves administering to the human subject (wherein the subject is an ApoE4 non-carrier or an ApoE4 carrier), multiple doses of the anti-beta-amyloid antibody, in order, beginning with step (a) performing two or more of the following administering steps prior to the onset of the ARIA:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 6 mg/kg of body weight of the subject;

(f) 4 weeks after step (e), administering the antibody to the subject in an amount of 6 mg/kg of body weight of the subject; and

(g) in consecutive intervals of 4 weeks after step (f), administering the antibody to the subject in an amount of 10 mg/kg of body weight of the subject.

In certain embodiments, the method comprises, after resolution of the ARIA, performing from the following administering steps, in order, those steps that were not performed prior to the onset of the ARIA:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 6 mg/kg of body weight of the subject;

(f) 4 weeks after step (e), administering the antibody to the subject in an amount of 6 mg/kg of body weight of the subject; and

(g) in consecutive intervals of 4 weeks after step (f), administering the antibody to the subject in an amount of 10 mg/kg of body weight of the subject.

In certain embodiments, administering to the human subject (wherein the subject is an ApoE4 carrier), multiple doses of the anti-beta-amyloid antibody comprises:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject; and

(c) in consecutive intervals of 4 weeks after step (b), administering the antibody to the subject in an amount of 3 mg/kg of body weight of the subject.

In some embodiments, wherein, after resuming administration of the anti-beta-amyloid antibody, the human subject develops a second ARIA. The second ARIA, can be, e.g.: (i) ARIA-E that is moderate or severe and is accompanied by no clinical symptoms; (ii) ARIA-E that is mild, moderate, or severe and is accompanied by mild, moderate, or severe clinical symptoms, or clinical symptoms meeting the “other medically important” serious criteria; (iii) ARIA-H with 5 to 9 cumulative microhemorrhages and accompanied by no clinical symptoms; (iv) ARIA-H with 1 to 9 cumulative microhemorrhages and accompanied by mild, moderate, or severe clinical symptoms, or clinical symptoms meeting the “other medically important” serious criteria; (v) ARIA-H with 2 cumulative areas of superficial siderosis and accompanied by no clinical symptoms; or (vi) ARIA-H with 1 or 2 cumulative areas of superficial siderosis and accompanied by mild, moderate, or severe clinical symptoms, or clinical symptoms meeting the “other medically important” serious criteria. The method further comprises suspending administration of the anti-beta-amyloid antibody to the subject until the second ARIA resolves (and clinical symptoms, if any, resolve). The method further involves resuming administration of the anti-beta-amyloid antibody to the subject at a dose that is lower than the dose that was administered to the subject immediately prior to the subject developing the second ARIA.

In some embodiments, the ARIA is accompanied by no clinical symptoms. In other embodiments, the ARIA is accompanied by mild clinical symptoms. In yet other embodiments, the ARIA is accompanied by moderate clinical symptoms. In still other embodiments, the ARIA is accompanied by clinical symptoms meeting the “other medically important” serious criteria.

In another aspect, this disclosure features a method for treating AD in a human subject in need thereof. The method involves administering to the human subject (wherein the subject is an ApoE4 carrier or ApoE4 non-carrier), multiple doses of an anti-beta-amyloid antibody (e.g., aducanumab). The method comprises:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 3 mg/kg of body weight of the subject;

(f) 4 weeks after step (e), administering the antibody to the subject in an amount of 3 mg/kg of body weight of the subject;

(g) 4 weeks after step (f), administering the antibody to the subject in an amount of 6 mg/kg of body weight of the subject;

(h) 4 weeks after step (g), administering the antibody to the subject in an amount of 6 mg/kg of body weight of the subject;

(i) 4 weeks after step (h), administering the antibody to the subject in an amount of 6 mg/kg of body weight of the subject;

(j) 4 weeks after step (i), administering the antibody to the subject in an amount of 6 mg/kg of body weight of the subject;

(k) 4 weeks after step (j), administering the antibody to the subject in an amount of 6 mg/kg of body weight of the subject; and

(l) in consecutive intervals of 4 weeks after step (k), administering the antibody to the subject in an amount of 10 mg/kg of body weight of the subject.

In certain embodiments, the subject is administered the antibody in an amount of 10 mg/kg of body weight of the subject for at least two, three, four, five, six, seven, or eight consecutive intervals of 4 weeks.

In another aspect, this disclosure features a method for treating AD in a human subject in need thereof. The method involves administering to the human subject (wherein the subject is an ApoE4 carrier or ApoE4 non-carrier), multiple doses of an anti-beta-amyloid antibody (e.g., aducanumab). The method comprises:

(a) administering the anti-beta-amyloid antibody to the subject in an amount of 3 mg/kg of body weight of the subject;

(b) 4 weeks after step (a), administering the antibody to the subject in an amount of 3 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 6 mg/kg of body weight of the subject;

(f) 4 weeks after step (e), administering the antibody to the subject in an amount of 6 mg/kg of body weight of the subject;

(g) 4 weeks after step (f), administering the antibody to the subject in an amount of 6 mg/kg of body weight of the subject;

(h) 4 weeks after step (g), administering the antibody to the subject in an amount of 6 mg/kg of body weight of the subject;

(i) 4 weeks after step (h), administering the antibody to the subject in an amount of 6 mg/kg of body weight of the subject; and

(j) in consecutive intervals of 4 weeks after step (i), administering the antibody to the subject in an amount of 10 mg/kg of body weight of the subject.

In certain embodiments, the subject is administered the antibody in an amount of 10 mg/kg of body weight of the subject for at least two, three, four, five, six, seven, or eight consecutive intervals of 4 weeks.

In another aspect, this disclosure features a method for treating AD in a human subject in need thereof. The method involves administering to the human subject (wherein the subject is an ApoE4 carrier or ApoE4 non-carrier), multiple doses of an anti-beta-amyloid antibody (e.g., aducanumab). The method comprises:

(a) administering the anti-beta-amyloid antibody to the subject in an amount of 1 mg/kg of body weight of the subject;

(b) 4 weeks after step (a), administering the antibody to the subject in an amount of 1 mg/kg of body weight of the subject;

(c) 4 weeks after step (b), administering the antibody to the subject in an amount of 3 mg/kg of body weight of the subject;

(d) 4 weeks after step (c), administering the antibody to the subject in an amount of 3 mg/kg of body weight of the subject;

(e) 4 weeks after step (d), administering the antibody to the subject in an amount of 6 mg/kg of body weight of the subject;

(f) 4 weeks after step (e), administering the antibody to the subject in an amount of 6 mg/kg of body weight of the subject; and

(g) in consecutive intervals of 4 weeks after step (f), administering the antibody to the subject in an amount of 10 mg/kg of body weight of the subject.

In certain embodiments, the subject is administered the antibody in an amount of 10 mg/kg of body weight of the subject for at least two, three, four, five, six, seven, or eight consecutive intervals of 4 weeks.

In another aspect, this disclosure features a method for treating AD in a human subject in need thereof. The method involves administering to the human subject (wherein the subject is an ApoE4 carrier or ApoE4 non-carrier), multiple doses of an anti-beta-amyloid antibody (e.g., aducanumab). The method comprises:

(a) administering the anti-beta-amyloid antibody to the subject in an amount of 3 mg/kg of body weight of the subject;

(b) 4 weeks after step (a), administering the antibody to the subject in an amount of 3 mg/kg of body weight of the subject;

(c) 4 weeks after step (b), administering the antibody to the subject in an amount of 6 mg/kg of body weight of the subject;

(d) 4 weeks after step (c), administering the antibody to the subject in an amount of 6 mg/kg of body weight of the subject; and

(e) in consecutive intervals of 4 weeks after step (d), administering the antibody to the subject in an amount of 10 mg/kg of body weight of the subject.

In certain embodiments, the subject is administered the antibody in an amount of 10 mg/kg of body weight of the subject for at least two, three, four, five, six, seven, or eight consecutive intervals of 4 weeks.

The following embodiments apply to all of the above aspects:

In certain embodiments, the anti-beta-amyloid antibody is administered to the human subject intravenously.

In some embodiments, the anti-beta-amyloid antibody comprises a heavy chain variable region (VH) and a light chain variable region (VL), wherein the VH comprises a first complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8. In certain instances, the antibody comprises a human IgG1 constant region.

In some embodiments, the anti-beta-amyloid antibody comprises a heavy chain variable region (VH) and a light chain variable region (VL), wherein the VH consists of SEQ ID NO:1 and the VL consists of SEQ ID NO:2. In certain instances, the antibody comprises a human IgG1 constant region.

In certain embodiments, the anti-beta-amyloid antibody comprises a heavy chain and a light chain, wherein the heavy chain consists of SEQ ID NO:10 and the light chain consists of SEQ ID NO:11.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 shows the mean positron emission tomography (PET) composite standardized uptake ratio values (SUVR) by time point as determined by PET scans in a study of subjects treated with antibody BIIB037.

FIG. 2 shows the adjusted mean change from baseline PET composite SUVR of the subjects by baseline clinical stage, namely, prodromal or mild AD.

FIG. 3 shows the adjusted mean change from baseline PET composite SUVR by baseline ApoE4 status of the subjects.

FIG. 4 reports the estimated incidence of ARIA-E and/or ARIA-H in a study of AD subjects treated with antibody BIIB037.

FIG. 5 shows the adjusted mean change from baseline Clinical Dementia Rating Sum of Boxes (CDR-SB) for patients dosed every 4 weeks for 54 weeks with placebo, or 1 mg/kg, 3 mg/kg, or 10 mg/kg of antibody BIIB037.

FIG. 6 shows the adjusted mean change from baseline Mini Mental State Examination (MMSE)+standard error (SE) for patients dosed every 4 weeks for 54 weeks with placebo, or 1 mg/kg, 3 mg/kg, or 10 mg/kg of antibody BIIB037.

FIGS. 7A-7F show amyloid plaque reduction with aducanumab. FIG. 7A shows mean composite SUVR over time for PD analysis population. The dashed line indicates the SUVR cut-point for florbetapir. FIGS. 7B-7F show adjusted mean (±SE) change from baseline in composite SUVR at 26 and 54 weeks among (FIG. 7B) the overall PD analysis population, (FIG. 7C) ApoE ε4 carriers, (FIG. 7D) non-carriers, and patients with (FIG. 7E) prodromal, and (FIG. 7F) mild AD.

FIG. 8 shows the effect of aducanumab on MMSE.

FIG. 9 shows the effect of aducanumab on CDR-SB.

FIG. 10 depicts selected dosing schedules for ApoE4 carriers and non-carriers.

FIG. 11 demonstrates the ability of aducanumab to reduce amyloid plaque.

FIG. 12 demonstrates a slowing of decline on CDR-SB with aducanumab.

FIG. 13 demonstrates a slowing of decline on MMSE with aducanumab.

FIG. 14 depicts the study design for PRIME, a multicenter, randomized, double-blind, placebo-controlled, multidose study. Patients (planned N=188) were randomized to 1 of 9 treatment arms (target enrollment: n=30 per active treatment arm) in a staggered, ascending dose design at a ratio of 3:1 active vs. placebo.

FIG. 15 depicts primary and secondary endpoints for the PRIME study.

FIG. 16 provides the PRIME assessment timeline. Data were analyzed to Week 54 for the 1, 3, and 10 mg/kg arms and to Week 30 for the 6 mg/kg arm.

FIG. 17 depicts patient disposition in the PRIME study. Of the 166 patients randomized, 165 were dosed; 107 (65%) were ApoE ε4 carriers, and 68 (41%) had prodromal AD.

FIG. 18 depicts baseline demographic and disease characteristics for the PRIME study.

FIG. 19 provides a summary of ARIA findings and patient disposition following ARIA-E.

### DETAILED DESCRIPTION

### Alzheimer's Disease

Alzheimer's disease, abbreviated herein as AD, is a dementia that is primarily identified by clinical diagnosis and established by markers of the disease.

AD is a continuum having certain operationally defined stages of disease progression. AD pathology begins prior to the onset of clinical symptoms. For example, amyloid plaques, one marker of AD pathology, form 10-20 years prior to the onset of AD dementia. The currently recognized stages of AD include preclinical, prodromal, mild, moderate, and severe. These stages may be further divided into subcategories based on the severity of symptoms and measures of AD progression.

Because AD does not occur in discrete stages, those skilled in the art will recognize that the differences between patient groups may not be distinct in a particular clinical setting. Nevertheless, the clinical disease stage can be characterized by measures, and changes in these measures over time, such as Aβ accumulation (CSF/PET), synaptic dysfunction (FDG-PET/fMRI), tau-mediated neuronal injury (CSF), brain structure (volumetric MRI), cognition, and clinical function. (Jack C R, et al. Hypothetical model of dynamic biomarkers of the Alzheimer's pathological cascade. Lancet Neurol., 2010; 9(1):119-28).

Current core clinical criteria for all dementia, referred to as the NINCDS-ADRDA criteria (McKhann G M, V. diagnosis of dementia due to Alzheimer's disease: Recommendations from the National Inst. on Aging-Alzheimer's Association workgroups on diagnostic guidelines for Alzheimer's disease. Alzheimer's & Dementia, 7 (2011) 263-269), are known in the art and can be employed in practicing this invention. They include cognitive or behavioral impairment involving impaired ability to acquire and remember new information, impaired reasoning and handling of complex tasks, impaired visuospatial abilities, impaired language functions (speaking, reading, writing), and changes in personality, behavior, or comportment. Alzheimer's disease is currently diagnosed using the core criteria and is typically characterized by symptoms which have a gradual onset over months to years, not sudden over hours or days (insidious onset). There is usually a clear-cut history of worsening of cognition by report or observation in Alzheimer's disease subjects.

Other diagnostic classification systems have evolved as new information on AD has become available. These systems include the International Working Group (IWG) new research criteria for diagnosis of AD (Dubois B et al., Lancet Neurol., 2007; 6(8):734-736), IWG research criteria, (Dubois et al., Lancet Neurol., 2010; 9(11):1118-27), NIA/AA Criteria (Jack C R et al. Alzheimer's Dement., 2011; 7(3):257-62), and DSM-5 criteria (American Psychiatric Association, DSM-5, 2013). These classification systems can also be employed in diagnosing AD subjects for treatment according to the methods of this disclosure.

### Patients

The term “patient” is meant to include any human subject for whom diagnosis, prognosis, prevention, or therapy for Alzheimer's disease is desired, and includes a human subject in need of treatment. Those in need of treatment include those already with AD, as well as those prone to have AD, or those in which the manifestation of AD is to be prevented. Typical patients will be men or women aged 40 to 90 (e.g., 45 to 90, 50 to 90, 55 to 90, 60 to 90). In one embodiment, the disclosure provides a method of treating a patient with AD (including, without limitation, patients with preclinical, prodromal, mild, moderate, or severe AD). In a further embodiment, the patient has amyloid pathology confirmed, e.g., by positron emission tomography (PET) imaging.

AD patients in need of treatment range from subjects with amyloid pathology and early neuronal degeneration to subjects with widespread neurodegeneration and irreversible neuronal loss with progressive cognitive and functional impairment to subjects with dementia.

Patients with preclinical AD can be identified by asymptomatic stages with or without memory complaints and emerging episodic memory and executive function deficits. This stage is typically characterized by the appearance of in vivo molecular biomarkers of AD and the absence clinical symptoms.

Prodromal AD patients are pre-dementia stage characterized predominantly by cognitive deficits and emerging functional impairment with disease progression. Prodromal AD patients typically have mini-mental state examination (MMSE) scores between 24-30 (inclusive), a spontaneous memory complaint, objective memory loss defined as a free recall score of <27 on the Free and Cued Selective Reminding Test (FCSRT), a global Clinical Dementia Rating (CDR) score of 0.5, absence of significant levels of impairment in other cognitive domains, and essentially preserved activities of daily living, and an absence of dementia.

Patients with mild AD typically have MMSE scores between 20-26 (inclusive), a global CDR of 0.5 or 1.0, and meet the National Institute on Aging-Alzheimer's Association core clinical criteria for probable AD (see Section 22).

Basing AD diagnosis on clinical symptoms, mild stage AD patients will exhibit conspicuous behavior at work, forgetfulness, mood swings, and attention disturbances. Moderate stage AD patients will exhibit cognitive deficits, restricted everyday activities, orientation disturbance, apraxia, agnosia, aphasia, and behavioral abnormalities. Severe stage AD patients are characterized by loss of independence, decay of memory and speech, and incontinence,

In certain embodiments, treatment is of earlier-stage patients who are amyloid positive as assessed by 18F-AV-45 PET scans. The patient may be asymptomatic for, or exhibit only transient symptoms of, headache, confusion, gait difficulties, or visual disturbances. The patient may or may not be an ApoE4 carrier as determined by ApoE genotyping.

In other embodiments, treatment is of patients having any medical or neurological condition (other than AD) that might be a contributing cause of the subject's cognitive impairment, such as stroke or other cerebrovascular condition, other neurodegenerative disease, a history of clinically significant psychiatric illness, acute or sub-acute micro- or macro hemorrhage, prior macrohemorrhage, or superficial siderosis. These patients can be treated following screening and selection by a qualified clinician.

Treatment

As used herein, the terms “treat” or “treatment” generally mean obtaining a desired pharmacological and/or physiological effect. The effect can be prophylactic in terms of completely or partially preventing AD or symptoms thereof and/or can be therapeutic in terms of partially or completely curing AD and/or one or more adverse effects attributed to AD. Hence, the term “treatment” as used herein includes: (a) preventing AD from occurring in a subject who may be predisposed to AD, but has not yet been diagnosed as having it; (b) inhibiting AD, e.g. arresting its development; (c) relieving AD, e.g. causing regression of AD; or (d) prolonging survival as compared to expected survival if not receiving treatment.

In one embodiment, the treatment is prophylactic for completely or partially preventing AD or symptoms thereof in the patient, or the treatment is therapeutic for partially or completely curing AD or symptoms attributed to AD in the patient.

In another embodiment, treatment has a disease modifying effect. This means that the treatment slows or delays the underling pathological or pathophysiological disease processes and there is an improvement in clinical signs and symptoms of AD relative to placebo.

In a further embodiment, treatment results in symptomatic improvement. This may consist of enhanced cognition, more autonomy, and/or improvement in neuropsychiatric and behavioral dysfunction, even if for only a limited duration.

While the goal of any therapy is the prevention or cure of disease, it will be understood that this disclosure contemplates a delay of clinical decline or progression of disease or relief of symptoms. Delaying clinical decline or disease progression directly impacts the patient and care-givers. It delays disability, maintains independence, and allows the patient to live a normal life for a longer period of time. Relief of symptoms to the best degree possible can incrementally improve cognition, function, and behavioral symptoms, as well as mood.

In the method of treatment of AD according to this disclosure, an anti-beta amyloid antibody is administered to the human patient. In one embodiment, the anti-beta amyloid antibody is a monoclonal antibody. In another embodiment, the anti-beta amyloid antibody is a fully human antibody. In a further embodiment, the anti-beta amyloid antibody is a recombinant antibody. In another embodiment, the anti-beta amyloid antibody is a recombinant, fully human, monoclonal antibody. In certain embodiments, the anti-beta amyloid antibody is selective for soluble Aβ oligomer and fibril binding without substantial monomer binding. These properties improve pharmacokinetics (PK), reduce antibody sink, and minimize off-target cross-reactivity with APP-expressing tissues. An exemplary monoclonal antibody meeting these criteria is antibody BIIB037.

Antibody BIIB037, also known as aducanumab, is a biologic treatment for Alzheimer's disease. It is an anti-Aβ antibody that recognizes aggregated forms of Aβ, including plaques. BIIB037 contains a human kappa light chain. BIIB037 consists of 2 heavy and 2 human kappa light chains connected by inter-chain disulfide bonds. By “BIIB037” or “aducanumab” is meant an anti-Aβ antibody comprising the amino acid sequences set forth in SEQ ID NOs.: 10 and 11.

In vitro characterization studies have established that antibody BIIB037 recognizes a conformational epitope present in Aβ aggregates, the accumulation of which is believed to underlie the development and progression of AD.

In vivo pharmacology studies indicate that a murine IgG2a chimeric version of the antibody (ch 12F6A) with similar properties significantly reduces amyloid plaque burden in the brains of aged Tg2576 mice, a mouse model of AD. The reduction in parenchymal amyloid was not accompanied by a change in vascular amyloid, as has been reported for certain anti-Aβ antibodies (Wilcock O M, Colton C A Immunotherapy, vascular pathology, and microhemorrhages in transgenic mice. CNS & Neurological Disorders Drug Targets, 2009 March; 8(1):50-64).

The VH and VL of antibody BIIB037 have amino acid sequences that are identical to the amino acid sequence of the VH and VL of antibody NI-101.12F6A described in U.S. Pat. No. 8,906,367 (see, Tables 2-4; incorporated by reference in its entirety herein). Specifically, antibody BIIB037 has an antigen binding domain comprising VH and VL variable regions depicted in Table 1 (VH) and Table 2 (VL), corresponding complementarity determining regions (CDRs) depicted in Table 3, and heavy and light chains depicted in Table 4 (H) and Table 5 (L).

TABLE 1
Amino acid sequences of the V_{H}
region of anti-Aβ antibody BIIB037
(VH CDRs (Kabat definition) underlined).
Variable heavy chain sequence

QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSS (SEQ ID NO: 1)

TABLE 2
Amino acid sequences of the V_{L}
region of anti-Aβ antibody BIIB037
(VL CDRs (Kabat definition) underlined).
Variable light chain sequence (kappa or lambda)

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKR (SEQ ID NO: 2)

TABLE 3
Denomination of CDR protein sequences in
Kabat Nomenclature of V_{H} and V_{L} regions
of anti-Aβ antibody BIIB037.

| | Variable | Variable |
|---|---|---|
| CDR | heavy chain | light chain |
| CDR1 | SYGMH | RASQSISSYLN |
| | (SEQ ID NO: 3) | (SEQ ID NO: 6) |
| CDR2 | VIWFDGTKKYYTDSVKG | AASSLQS |
| | (SEQ ID NO: 4) | (SEQ ID NO: 7) |
| CDR3 | DRGIGARRGPYYMDV | QQSYSTPLT |
| | (SEQ ID NO: 5) | (SEQ ID NO: 8) |

The amino acid sequence of the mature heavy chain of BIIB037 is provided in Table 4 below.

TABLE 4
Amino acid sequences of the
heavy chain of anti-Aβ antibody BIIB037
(heavy chain CDRs (Kabat definition) underlined).
Heavy chain sequence

QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK RVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGFY PSDIAVEWES NGQPENNYKT
TPPVLDSDGS FFLYSKLTVD KSRWQQGNVF SCSVMHEALH
NHYTQKSLSL SPG (SEQ ID NO: 10)

The amino acid sequence of the mature light chain of BIIB037 is provided in Table 5 below.

TABLE 5
Amino acid sequences of the
light chain of anti-Aβ antibody BIIB037
(light chain CDRs (Kabat definition) underlined).
Light chain sequence

DIQMTQSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC (SEQ ID NO: 11)

In addition to antibody BIIB037, this disclosure contemplates the use of the other anti-beta-amyloid antibodies, such as antibodies comprising either the VH region comprising or consisting of SEQ ID NO:1 or the VL region comprising or consisting of SEQ ID NO:2, or antibodies comprising the VH region comprising or consisting of SEQ ID NO:1 and the VL region comprising or consisting of SEQ ID NO:2, wherein the VH and/or VL regions have one or more substitutions, deletions, and/or insertions. In some embodiments, these VH and VL regions may have up to 25, up to 20, up to 15, up to 10, up to 5, or 1, 2, 3, 4, 5, 6, 7, 8, 9, 10, 11, 12, 13, 14, 15, 16, 17, 18, 19, or 20 amino acid substitutions and still bind beta-amyloid. In specific embodiments, these amino acid substitutions occur only in the framework region. In some embodiments, the amino acid substitution(s) is/are conservative amino acid substitutions. In certain embodiments, the VH and VL regions may include 1 to 5 (1, 2, 3, 4, 5) amino acid deletions and/or additions and still bind beta-amyloid. In certain embodiments, these deletions and/or additions are made at the N- and/or C-terminus of the VH and/or VL regions. In one embodiment, one amino acid is deleted and/or added at the N and/or C-terminus of the VH region. In one embodiment, one amino acid is deleted and/or added at the N and/or C-terminus of the VL region.

Other antibodies contemplated for use in the disclosure include antibodies comprising the variable heavy chain (VH) CDRs and the variable light chain (VL) CDRs in Table 3. Thus, the anti-beta amyloid antibodies comprise the CDRs comprising or consisting of the amino acid sequences of SEQ ID NOs.: 3-8. In one embodiment, the anti-beta amyloid antibodies comprise the CDRs comprising or consisting of the amino acid sequences of SEQ ID NOs.: 4-8 and include as VH CDR1 an amino acid sequence comprising or consisting of GFAFSSYGMH (SEQ ID NO:9). In some instances, the disclosure encompasses anti-beta-amyloid antibodies comprising the VH and VL CDRs of BIIB037 based on any CDR definition (e.g., Kabat, Chothia, enhanced Chothia, AbM, or contact definition). See, e.g., http://www.bioinf.org.uk/abs/index.html. In one embodiment, the disclosure encompasses anti-beta-amyloid antibodies comprising the VH and VL CDRs of BIIB037 based on the Chothia definition. In one embodiment, the disclosure encompasses anti-beta-amyloid antibodies comprising the VH and VL CDRs of BIIB037 based on the enhanced Chothia definition. In another embodiment, the disclosure encompasses anti-beta-amyloid antibodies comprising the VH and VL CDRs of BIIB037 based on the AbM definition. In yet another embodiment, the disclosure encompasses anti-beta-amyloid antibodies comprising the VH and VL CDRs of BIIB037 based on the contact definition.

Antibody BIIB037 and other antibodies employed in the invention can be prepared using known methods. In some embodiments, the antibody is expressed in a Chinese hamster ovary (CHO) cell line.

The patient's response to treatment according to the invention is generally dose-dependent. One embodiment of the invention comprises administering at least one dose of the anti-Aβ antibody to the patient in an amount that is less than the minimum therapeutic amount required to treat the patient for AD. This is followed by at least one dose of the anti-Aβ antibody administered to the patient in an amount that is about equal to the minimum therapeutic amount required to treat the patient for AD. And then at least one dose of the anti-Aβ antibody is administered to the patient in an effective amount that is more than the minimum therapeutic amount, but less than the maximum tolerated amount required to treat the patient for AD. In a preferred embodiment, cerebral amyloid burden is reduced. In a further preferred embodiment, the susceptibility of the patient to ARIA is reduced.

A therapeutically effective amount refers to the amount of the anti-Aβ antibody sufficient to ameliorate a symptom or condition associated with Alzheimer's disease. Therapeutic efficacy and toxicity of the anti-Aβ antibody can be determined by standard pharmaceutical procedures. Ideally, the anti-Aβ antibody is employed in an amount sufficient to restore normal behavior and/or cognitive properties in case of Alzheimer's disease, or at least delay or prevent the progression of AD in the patient.

In Tg2576 mice, a dose-dependent reduction in cerebral amyloid was observed after chronic dosing with monoclonal antibody BIIB037 (0.3 mg/kg to 30 mg/kg). A significant amyloid reduction was observed at 3 mg/kg, deemed the minimum therapeutic dose for antibody BIIB037 in this animal model.

An effective amount of the anti-Aβ antibody is that quantity of the antibody that will produce a clinically significant response in the treatment of Alzheimer's disease. Effective amounts of about 1 to 30 mg/kg per month (e.g., 1 mg/kg, 3 mg/kg, 6 mg/kg, 10 mg/kg, 12 mg/kg, 15 mg/kg, 18 mg/kg, 20 mg/kg, 24 mg/kg, 25 mg/kg, 28 mg/kg, 30 mg/kg) can be employed. Efficacy of antibody BIIB037 can reach a plateau at effective amounts between about 10 mg/kg and about 30 mg/kg of the patient's body weight, consistent with safety. In certain embodiments, an effective amount of about 3 mg/kg to about 10 mg/kg of the patient's body weight is contemplated. In other embodiments, effective amounts are about 3 mg/kg, about 6 mg/kg, and about 10 mg/kg of the patient's body weight.

The maximum tolerated amount of the anti-Aβ antibody is that quantity of the antibody which will produce a clinically significant response in the treatment of Alzheimer's disease consistent with safety. A principal safety concern in treating patients according to the method of the invention is the occurrence of ARIA, especially ARIA-E or ARIA-H. The methods of the invention make it possible to employ higher doses of antibody BIIB037 for the treatment of patients for AD than was feasible using previously known protocols.

It will be understood that dose adjustments can be implemented during the treatment protocol. For example, for reasons of safety or efficacy, doses can be increased so that the effects of the anti-Aβ antibody on AD can be enhanced or doses can be decreased so that the ARIA rate and severity can be mitigated. If a dose is missed, the patient should preferably resume dosing by receiving the missed dose and continuing thereafter according to the described regimen.

In certain embodiments, the anti-Aβ antibody is administered to the patient by intravenous infusion following dilution into saline. When using this mode of administration, each infusion step in the titration regime of the invention will typically take about 1 hour.

The dose ranges and other numerical values herein include a quantity that has the same effect as the numerically stated amount as indicated by treatment of Alzheimer's disease in the patient and a reduction in the incidence or susceptibility of the patient to ARIA when compared to an individual not treated by the method of the invention. At the very least, each numerical parameter should be construed in light of the number of significant digits, applying ordinary rounding techniques. In addition, any numerical value inherently contains certain errors from the standard deviation of its measurement and such values are within the scope of the invention.

### Compositions

An anti-Aβ antibody described herein (e.g., BIIB037) can be formulated as a pharmaceutical composition. The pharmaceutical compositions employed in the present invention can be formulated according to methods well known in the art; see, for example, Remington: The Science and Practice of Pharmacy (2000) by the University of Sciences in Philadelphia, ISBN 683-306472. The compositions can further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc.

Furthermore, the pharmaceutical composition may comprise additional agents. For example, for use in the treatment of Alzheimer's disease the additional agent can be selected from the group consisting of a small organic molecule, another anti-Aβ antibody, an anti-Tau antibody, and combinations thereof. Non-limiting examples of anti-Aβ antibodies can be found in U.S. Pat. No. 8,906,367. Non-limiting examples of anti-Tau antibodies can be found in U.S. Pat. No. 8,940,272 and US Patent Application Publication US 2015/0344553.

Administration of the compositions can be effected in different ways, e.g., by intravenous, intraperitoneal, subcutaneous, intramuscular, topical, or intradermal administration.

### Standard Doses

In one method of treatment of Alzheimer's disease, an anti-beta amyloid antibody (e.g., BIIB037) is administered to a human patient in multiple doses (e.g., 1, 2, 3, 4, 5, 6, 7, 8, 9, 10, 11, 12) of the same amount of antibody (i.e., a standard dose) over a period of time.

For example, the human patient may be administered 3 mg/kg of the patient's body weight of the anti-beta amyloid antibody on multiple occasions over a period of time.

In another example, the human patient may be administered 6 mg/kg of the patient's body weight of the anti-beta amyloid antibody on multiple occasions over a period of time.

In another example, the human patient may be administered 10 mg/kg of the patient's body weight of the anti-beta amyloid antibody on multiple occasions over a period of time.

In yet another example, the human patient may be administered 15 mg/kg of the patient's body weight of the anti-beta amyloid antibody on multiple occasions over a period of time.

In a further example, the human patient may be administered 20 mg/kg of the patient's body weight of the anti-beta amyloid antibody on multiple occasions over a period of time.

In another example, the human patient may be administered 30 mg/kg of the patient's body weight of the anti-beta amyloid antibody on multiple occasions over a period of time.

The period of time for each of these methods can be, e.g., once every 1, 2, 3, 4, 5, 6, 7, or 8 weeks. The treatment can proceed until such time as deemed beneficial by a health care practitioner.

In certain embodiments, the anti-Aβ antibody is administered to the patient by intravenous infusion following dilution into saline.

In any of the above embodiments, the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8.

In some embodiments, the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition.

In some embodiments, the anti-Aβ antibody comprises a VH and a VL, wherein the VH comprises or consists of SEQ ID NO:1 and the VL comprises or consists of SEQ ID NO:2.

In certain embodiments, the anti-Aβ antibody described above further comprises a human IgG1 constant region.

In a particular embodiment, the anti-Aβ antibody comprises a heavy chain comprising or consisting of SEQ ID NO:10 and a light chain comprising or consisting of SEQ ID NO:11.

### Titration (Sequential Administration of Increasing Doses)

Occurrence of the ARIA in AD patients treated with anti-beta-amyloid antibodies (e.g., BIIB037) is dose-dependent. ARIA has been observed in patients receiving 1 mg/kg and 3 mg/kg of the antibody after the third and fifth doses. At doses of 6 mg/kg and 10 mg/kg of body weight, ARIA has been observed after the second dose. The methods of the disclosure include treatment regimens selected to decrease the incidence of ARIA.

In one method of treatment of Alzheimer's disease, the anti-beta amyloid antibody is administered to a human patient in increasing amounts over a period of time. This procedure of sequentially administering the antibody to the patient is referred to herein as “titration” because it involves administering a standardized pharmaceutical of known concentrations in carefully measured amounts until completion of the procedure as evidenced by specific endpoints. In the present invention, the endpoints include the effect of the treatment on Alzheimer's disease in the patient and the effect of the treatment in reducing the incidence of ARIA, especially ARIA-E or ARIA-H, in the treated patient population.

One of the advantages of the titration regime of the invention is that it makes it possible to administer higher doses of the monoclonal antibody to AD patients, especially apolipoprotein ε4 (ApoE4) carriers, without incurring the same extent of ARIA observed with a standard dose regimen. In certain embodiments, the higher dose comprises a dose or doses of the anti-Aβ antibody of 10 mg/kg of the body weight of the subject. Without intending to be limited to any particular mechanism, it is believed that titration results in lower initial amyloid removal and slower removal during the overall treatment.

Titration of the anti-Aβ antibody (e.g., BIIB037) is carried out in multiple doses. For example, two doses of the antibody can be administered to the patient in an amount per dose that is less than the minimum therapeutic amount, followed by 4 doses of the antibody in an amount per dose that is about equal to the minimum therapeutic amount. This regime can then be followed by multiple doses in an amount per dose that is more than the minimum therapeutic amount, but less than the maximum tolerated amount until there is an acceptable change in AD in the patient. For example, doses can be administered approximately 4 weeks apart over approximately 52 weeks (a total of 14 doses). Progress can be monitored by periodic assessment.

One protocol of the disclosure, designated Protocol (1), comprises:

(A) administering the anti-beta amyloid antibody to the patient in an amount of 1 mg/kg of body weight of the patient;

(B) 4 weeks after step (A), administering the antibody to the patient in an amount of 1 mg/kg of body weight of the patient;

(C) 4 weeks after step (B), administering the antibody to the patient in an amount of 3 mg/kg of body weight of the patient;

(D) 4 weeks after step (C), administering the antibody to the patient in an amount of 3 mg/kg of body weight of the patient;

(E) 4 weeks after step (D), administering the antibody to the patient in an amount of 3 mg/kg of body weight of the patient;

(F) 4 weeks after step (E), administering the antibody to the patient in an amount of 3 mg/kg of body weight of the patient;

(G) 4 weeks after step (F), administering the antibody to the patient in an amount of 6 mg/kg of body weight of the patient; and

(H) in consecutive intervals of 4 weeks after step (G), administering the antibody to the patient in an amount of 6 mg/kg of body weight of the patient.

In other words, Protocol (1) comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, doses 3, 4, 5, and 6 of the antibody are administered to the patient in an amount of 3 mg/kg of body weight. And then, in four week intervals after administration of dose 6, doses 7 and 8 of the antibody are administered to the patient in an amount of 6 mg/kg of body weight.

Protocol (1) may comprise a total of 14 doses administered about 4 weeks apart over about 52 weeks, optionally continuing to dose about every 4 weeks thereafter, to thereby treat AD with reduced susceptibility of the patient to ARIA. In other words, four weeks after the administration of dose 8, doses 9-14 may be administered to the patient in an amount of 6 mg/kg body weight in four week intervals. In some embodiments, the antibody continues to be administered to the patient in an amount of 6 mg/kg of body weight every 4 weeks to at least week 76. In other words, in some embodiments, the method comprises administering doses 9-20 to the patient in an amount of 6 mg/kg body weight in four week intervals following dose 8. In some embodiments, after dose 8, the antibody is administered to the patient in an amount of 6 mg/kg of body weight every 4 weeks indefinitely. In some embodiments, in 12 week intervals following the last dose at 6 mg/kg body weight, the amount of antibody administered to the patient is 3 mg/kg body weight. In some embodiments, this reduced dose is initially administered to the patient 12 weeks after week 52 (i.e., 12 weeks after dose 14); in other embodiments, this reduced dose is administered to the patient 12 weeks after week 76 (i.e., 12 weeks after dose 20). In some embodiments, in four week intervals after the last dose at 6 mg/kg body weight, the amount of antibody administered to the patient is 1 mg/kg body weight. In some embodiments, this reduced dose is initially administered to the patient four weeks after week 52 (i.e., four weeks after dose 14); in other embodiments, this reduced dose is initially administered to the patient four weeks after week 76 (i.e., four weeks after dose 20).

Protocol (1) may be employed with patients designated as an ApoE4 carrier or an ApoE4 non-carrier as determined by ApoE genotyping. In any of the alternative embodiments of Protocol (1), the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8. In some embodiments of Protocol (1), the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition. In some embodiments of Protocol (1), the anti-Aβ antibody comprises a VH and a VL, wherein the VH comprises or consists of SEQ ID NO:1 and the VL comprises or consists of SEQ ID NO:2. In certain embodiments of Protocol (1), the anti-Aβ antibody comprises a human IgG1 constant region. In a particular embodiment, the anti-Aβ antibody comprises a heavy chain comprising or consisting of SEQ ID NO:10 and a light chain comprising or consisting of SEQ ID NO:11.

Another protocol according to the disclosure, designated Protocol (2), comprises:

(A) administering the anti-beta amyloid antibody to the patient in an amount of 1 mg/kg of body weight of the patient;

(B) 4 weeks after step (A), administering the antibody to the patient in an amount of 1 mg/kg of body weight of the patient;

(C) 4 weeks after step (B), administering the antibody to the patient in an amount of 3 mg/kg of body weight of the patient;

(D) 4 weeks after step (C), administering the antibody to the patient in an amount of 3 mg/kg of body weight of the patient;

(E) 4 weeks after step (D), administering the antibody to the patient in an amount of 6 mg/kg of body weight of the patient;

(F) 4 weeks after step (E), administering the antibody to the patient in an amount of 6 mg/kg of body weight of the patient; and

(G) in consecutive intervals of 4 weeks after step (F), administering the antibody to the patient in an amount of 10 mg/kg of body weight of the patient.

In other words, Protocol (2) comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, antibody doses 3 and 4 are administered to the patient in an amount of 3 mg/kg of body weight. In four week intervals after administration of dose 4, doses 5 and 6 of the antibody are administered to the patient in an amount of 6 mg/kg of body weight. And then, four weeks after administration of dose 6, antibody dose 7 is administered to the patient in an amount of 10 mg/kg of body weight.

Protocol (2) may comprise a total of 14 doses administered about 4 weeks apart over about 52 weeks, optionally continuing to dose about every 4 weeks thereafter, to thereby treat AD with reduced susceptibility of the patient to ARIA. In other words, four weeks after the administration of dose 7, doses 8-14 may be administered to the patient in an amount of 10 mg/kg body weight in four week intervals. In some embodiments, the anti-Aβ antibody continues to be administered to the patient in an amount of 10 mg/kg of body weight every 4 weeks to at least week 76. In other words, in some embodiments, the method comprises administering doses 8-20 to the patient in an amount of 10 mg/kg body weight in four week intervals following dose 7. In some embodiments, following dose 7, the anti-Aβ antibody is administered to the patient in an amount of 10 mg/kg of body weight every 4 weeks indefinitely. In some embodiments, after the last dose at 10 mg/kg body weight, the amount of anti-Aβ antibody is reduced to 3 mg/kg body weight and is administered to the patient in 12 week intervals. In some embodiments, this reduced dose is initially administered to the patient 12 weeks after week 52 (i.e., 12 weeks after dose 14); in other embodiments, this reduced dose is initially administered to the patient 12 weeks after week 76 (i.e., 12 weeks after dose 20). In some embodiments, four weeks after the last dose at 10 mg/kg body weight, the amount of antibody administered to the patient is reduced to 1 mg/kg body weight every 4 weeks. In some embodiments, this reduced dose begins four weeks after week 52 (i.e., four weeks after dose 14); in other embodiments, this reduced dose begins four weeks after week 76 (i.e., four weeks after dose 20).

Protocol (2) can be employed for the treatment of both ApoE4 carriers and ApoE4 non-carriers. In any of the alternative embodiments of Protocol (2), the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8. In some embodiments of Protocol (2), the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition. In certain embodiments of Protocol (2), the anti-Aβ antibody may comprise a heavy chain variable region (VH) and a light chain variable region (VL), wherein the VH comprises or consists of SEQ ID NO:1 and the VL comprises or consists of SEQ ID NO:2. In some embodiments of Protocol (2), the anti-Aβ antibody comprises a human IgG1 constant region. In a particular embodiment, the anti-Aβ antibody comprises a heavy chain comprising or consisting of SEQ ID NO:10 and a light chain comprising or consisting of SEQ ID NO:11.

This disclosure provides another protocol, designated Protocol (3), for the treatment of ApoE4 carriers. This embodiment comprises:

(A) administering the anti-beta amyloid antibody to the patient in an amount of 1 mg/kg of body weight of the patient;

(B) 4 weeks after step (A), administering the antibody to the patient in an amount of 1 mg/kg of body weight of the patient; and

(C) in consecutive intervals of 4 weeks after step (B), administering the antibody to the patient in an amount of 3 mg/kg of body weight of the patient.

In other words, Protocol (3) comprises administering a first dose of an anti-beta amyloid antibody to the patient in an amount of 1 mg/kg of body weight of the patient. Four weeks after the first dose, a second dose of the antibody is administered to the patient in an amount of 1 mg/kg of body weight. And then, 4 weeks after the second dose, dose 3 of the antibody is administered to the patient in an amount of 3 mg/kg of body weight.

Protocol (3) may comprise a total of 14 doses administered about 4 weeks apart over about 52 weeks, optionally continuing to dose about every 4 weeks thereafter, to thereby treat AD with reduced susceptibility of the patient to ARIA. In other words, four weeks after the administration of dose 3, doses 4-14 may be administered to the patient in an amount of 3 mg/kg body weight in four week intervals. In some embodiments, the antibody continues to be administered to the patient in an amount of 3 mg/kg of body weight every 4 weeks to at least week 76. In other words, in some embodiments, the method comprises administering doses 4-20 to the patient in an amount of 3 mg/kg body in four week intervals following dose 3. In some embodiments, following dose 3, the antibody is administered to the patient in an amount of 3 mg/kg of body weight every 4 weeks indefinitely. In some embodiments, after a prescribed period, the amount of antibody administered to the patient may be reduced to 3 mg/kg body weight every 12 weeks. In some embodiments, the 12 week dosing intervals begin after week 52 (i.e., after dose 14); in other embodiments, the 12 week dosing intervals begin after week 76 (i.e., after dose 20). In some embodiments, after a prescribed period, the amount of antibody administered to the patient may be reduced to 1 mg/kg body weight every 4 weeks. In some embodiments, this reduced dose begins four weeks after week 52 (i.e., four weeks after dose 14); in other embodiments, this reduced dose begins four weeks after week 76 (i.e., four weeks after dose 20).

Protocol (3) may be used with ApoE4 carriers as determined by ApoE genotyping. In any of the alternative embodiments of Protocol (3), the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8. In some embodiments of Protocol (3), the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition. In some embodiments of Protocol (3), the anti-Aβ antibody comprises a VH and a VL, wherein the VH comprises or consists of SEQ ID NO:1 and the VL comprises or consists of SEQ ID NO:2. In certain embodiments of Protocol (3), the anti-Aβ antibody comprises a human IgG1 constant region. In a particular embodiment, the anti-Aβ antibody comprises a heavy chain comprising or consisting of SEQ ID NO:10 and a light chain comprising or consisting of SEQ ID NO:11.

Another protocol of the disclosure, designated Protocol (4), comprises:

(A) administering the anti-beta amyloid antibody to the patient in an amount of 1 mg/kg of body weight of the patient;

(B) 4 weeks after step (A), administering the antibody to the patient in an amount of 1 mg/kg of body weight of the patient;

(C) 4 weeks after step (B), administering the antibody to the patient in an amount of 3 mg/kg of body weight of the patient;

(D) 4 weeks after step (C), administering the antibody to the patient in an amount of 3 mg/kg of body weight of the patient; and

(E) 4 weeks after step (D), administering the antibody to the patient in an amount of 6 mg/kg of body weight of the patient.

In other words, Protocol (4) comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, doses 3 and 4 are administered to the patient in an amount of 3 mg/kg of body weight. And then, four weeks after administration of dose 4, dose 5 of the antibody is administered to the patient in an amount of 6 mg/kg of body weight.

Protocol (4) may comprise a total of 14 doses administered about 4 weeks apart over about 52 weeks, optionally continuing to dose about every 4 weeks thereafter, to thereby treat AD with reduced susceptibility of the patient to ARIA. In other words, four weeks after the administration of dose 5, doses 6-14 may be administered to the patient in an amount of 6 mg/kg body weight in four week intervals. In some embodiments, the antibody continues to be administered to the patient in an amount of 6 mg/kg of body weight every 4 weeks to at least week 76. In other words, in some embodiments, the method comprises administering doses 6-20 to the patient in an amount of 6 mg/kg body weight in four week intervals following dose 5. In some embodiments, following dose 5, the antibody is administered to the patient in an amount of 6 mg/kg of body weight every 4 weeks indefinitely. In some embodiments, after the last dose at 6 mg/kg body weight, the amount of antibody administered to the patient is reduced to 3 mg/kg body weight every 12 weeks. In some embodiments, this reduced dose is initially administered to the patient 12 weeks after week 52 (i.e., 12 weeks after dose 14); in other embodiments, this reduced dose is initially administered to the patient 12 weeks after week 76 (i.e., 12 weeks after dose 20). In some embodiments, after the last dose at 10 mg/kg body weight, the amount of antibody administered to the patient is reduced to 1 mg/kg body weight every 4 weeks. In some embodiments, this reduced dose begins four weeks after week 52 (i.e., four weeks after dose 14); in other embodiments, this reduced dose begins four weeks after week 76 (i.e., four weeks after dose 20).

In any of the alternative embodiments of Protocol (4), the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8. In some embodiments of Protocol (4), the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition. In some embodiments of Protocol (4), the anti-Aβ antibody comprises a VH and a VL, wherein the VH comprises or consists of SEQ ID NO:1 and the VL comprises or consists of SEQ ID NO:2. In certain embodiments of Protocol (4), the anti-Aβ antibody comprises a human IgG1 constant region. In a particular embodiment, the anti-Aβ antibody comprises a heavy chain comprising or consisting of SEQ ID NO:10 and a light chain comprising or consisting of SEQ ID NO:11.

Yet another protocol of the disclosure, designated as Protocol (5), comprises: (A) administering the anti-beta amyloid antibody to the patient in an amount of 1 mg/kg of body weight of the patient;

(B) 4 weeks after step (A), administering the antibody to the patient in an amount of 1 mg/kg of body weight of the patient;

(C) 4 weeks after step (B), administering the antibody to the patient in an amount of 3 mg/kg of body weight of the patient;

(D) 4 weeks after step (C), administering the antibody to the patient in an amount of 3 mg/kg of body weight of the patient;

(E) 4 weeks after step (D), administering the antibody to the patient in an amount of 3 mg/kg of body weight of the patient;

(F) 4 weeks after step (E), administering the antibody to the patient in an amount of 3 mg/kg of body weight of the patient;

(G) in consecutive intervals of 4 weeks after step (F), administering the antibody to the patient in an amount of 6 mg/kg of body weight of the patient;

(H) in consecutive intervals of 4 weeks after step (G), administering the antibody to the patient in an amount of 6 mg/kg of body weight of the patient;

(I) in consecutive intervals of 4 weeks after step (H), administering the antibody to the patient in an amount of 6 mg/kg of body weight of the patient;

(J) in consecutive intervals of 4 weeks after step (I), administering the antibody to the patient in an amount of 6 mg/kg of body weight of the patient;

(K) in consecutive intervals of 4 weeks after step (J), administering the antibody to the patient in an amount of 6 mg/kg of body weight of the patient; and

(L) in consecutive intervals of 4 weeks after step (K), administering the antibody to the patient in an amount of 10 mg/kg of body weight of the patient.

In other words, Protocol (5) comprises administering a first dose of anti-beta amyloid antibody to the patient in an amount of 1 mg/kg of body weight of the patient, followed by a second dose in an amount of 1 mg/kg of body weight four weeks after the first dose. In four week intervals after the second dose, antibody doses 3, 4, 5, and 6 are administered to the patient in an amount of 3 mg/kg of body weight. In four week intervals after administration of dose 6, doses 7, 8, 9, 10, and 11 are administered to the patient in an amount of 6 mg/kg of body weight. And then, four weeks after administration of dose 11, dose 12 of the antibody is administered to the patient in an amount of 10 mg/kg of body weight.

Protocol (5) may comprise a total of 14 doses administered about 4 weeks apart over about 52 weeks, optionally continuing to dose about every 4 weeks thereafter, to thereby treat AD with reduced susceptibility of the patient to ARIA. In other words, four weeks after the administration of dose 12, doses 13-14 may be administered to the patient in an amount of 10 mg/kg body weight in four week intervals. In some embodiments, the antibody continues to be administered to the patient in an amount of 10 mg/kg of body weight every 4 weeks to at least week 76. In other words, in some embodiments, the method comprises administering doses 13-20 to the patient in an amount of 6 mg/kg body weight in four week intervals following dose 12. In some embodiments, following dose 12, the antibody is administered to the patient in an amount of 10 mg/kg of body weight every 4 weeks indefinitely. In some embodiments, after the last dose at 10 mg/kg body weight, the amount of antibody administered to the patient is reduced to 3 mg/kg body weight every 12 weeks. In some embodiments, this reduced dose is initially administered to the patient 12 weeks after week 52 (i.e., 12 weeks after dose 14); in other embodiments, this reduced dose is initially administered to the patient 12 weeks after week 76 (i.e., 12 weeks after dose 20). In some embodiments, after the last dose at 10 mg/kg body weight, the amount of antibody administered to the patient is reduced to 1 mg/kg body weight every 4 weeks. In some embodiments, this reduced dose begins four weeks after week 52 (i.e., four weeks after dose 14); in other embodiments, this reduced dose begins four weeks after week 76 (i.e., four weeks after dose 20). In certain embodiments, the subject being administered under Protocol (5) is an ApoE4 carrier. Higher doses (such as 10 mg/kg) of aducanumab can be administered in a titration regimen in ApoE4 carriers without incurring the same extent of ARIA observed with a fixed-dose regimen. In other embodiments, the subject being administered under Protocol (5) is an ApoE4 non-carrier.

In any of the alternative embodiments of Protocol (5), the anti-Aβ antibody may comprise a heavy chain variable region (VH) and a light chain variable region (VL), wherein the VH comprises a first complementarity determining region (VHCDR1) with the amino acid sequence SEQ ID NO:3 or SEQ ID NO:9, a VHCDR2 with the amino acid sequence SEQ ID NO:4, and a VHCDR3 with the amino acid sequences SEQ ID NO:5, and wherein the VL comprises a VLCDR1 with the amino acid sequence SEQ ID NO:6, a VLCDR2 with the amino acid sequence SEQ ID NO:7, and a VLCDR3 with the amino acid sequence SEQ ID NO:8. In some embodiments of Protocol (5), the anti-Aβ antibody comprises a VH and a VL, wherein the VH comprises VH CDR1, VH CDR2, and VH CDR3 of SEQ ID NO:1; and wherein the VL comprises VL CDR1, VL CDR2, and VLCDR3 of SEQ ID NO:2, wherein the CDRs are defined based on Chothia, enhanced Chothia, AbM, or the contact definition. In some embodiments of Protocol (5), the anti-Aβ antibody comprises a VH and a VL, wherein the VH comprises or consists of SEQ ID NO:1 and the VL comprises or consists of SEQ ID NO:2. In certain embodiments of Protocol (5), the anti-Aβ antibody comprises a human IgG1 constant region. In a particular embodiment, the anti-Aβ antibody comprises a heavy chain comprising or consisting of SEQ ID NO:10 and a light chain comprising or consisting of SEQ ID NO:11.

Exemplary dosing schemes for ApoE4 carriers and ApoE4 non-carriers are described in Table 6 below:

TABLE 6
Dosing Scheme for Anti-Aβ Antibody by Regimen

| | | Dose (Month) | | | | | | |
|---|---|---|---|---|---|---|---|---|
| | | 1 | 2 | 3 | 4 | 5 | 6 | 7 to 20 |
| Regimen | Dose (mg/kg) | | | | | | | |
| ApoE | Low Dose | 1 | 1 | 3 | 3 | 3 | 3 | 3 |
| ε4(+) | High Dose 1 | 1 | 1 | 3 | 3 | 3 | 3 | 6 |
| | High Dose 2 | 1 | 1 | 3 | 3 | 6 | 6 | 10 |
| | Placebo | saline | | | | | | |
| ApoE | Low Dose | 1 | 1 | 3 | 3 | 3 | 3 | 6 |
| ε4(−) | High Dose | 1 | 1 | 3 | 3 | 6 | 6 | 10 |

| | |
|---|---|
| Placebo | saline |

The exemplary protocols discussed above optimize efficacy with safety requirements. In certain embodiments of the invention, the patient's susceptibility to vasogenic edema (VE) is reduced, or the patient's susceptibility to cerebral microhemorrhages (mH) is reduced, or both VE and mH are reduced in the patient.

Variations of these preferred protocols are also possible. A dosing scheme of multiple doses of 1 mg/kg of the patient's body weight of the anti-Aβ antibody at periodic intervals between doses, followed by multiple doses of 3 mg/kg at periodic intervals between doses can be employed. For example, a dosing scheme comprises 2 doses of 1 mg/kg of the patient's body weight at intervals of 4 weeks between doses, followed by 4 doses of 3 mg/kg at intervals of 4 weeks between doses. Another example of this dosing scheme comprises 2 doses of 1 mg/kg of the patient's body weight at intervals of 4 weeks between doses, followed by multiple doses of 3 mg/kg at intervals of 4 weeks between doses until treatment is terminated. Another example of this dosing scheme comprises 4 doses of 1 mg/kg of the patient's body weight at intervals of 4 weeks between doses, followed by multiple doses of 3 mg/kg at intervals of 4 weeks between doses until treatment is terminated. Given that ARIA generally occurs between doses 2 and 5, this abbreviated protocol can provide an additional margin of safety. Thus, it may not be necessary for patients to continue to titrate to 6 mg/kg, but rather escalation of the dose can be stopped at about 3 mg/kg of the patient's body weight.

Another variation of these preferred protocols comprises a dosing scheme of multiple doses of 1 mg/kg of the patient's body weight of the anti-Aβ antibody at periodic intervals between doses, followed by multiple doses of 3 mg/kg at periodic intervals between doses can be employed, and finally multiple doses of 6 mg/kg of the patient's body weight at periodic intervals between doses until treatment is terminated. An example of this dosing scheme comprises 2 doses of 1 mg/kg of the patient's body weight at intervals of 4 weeks between doses, followed by 4 doses of 3 mg/kg at intervals of 4 weeks between doses can be employed, and finally multiple doses of 6 mg/kg of the patient's body weight until the treatment is terminated.

In another embodiment, an exemplary dosing scheme begins with dosing at 3 mg/kg of the patient's body weight at intervals of 4 weeks between doses (e.g., 2 doses, 4 doses, 5 doses), followed by multiple doses (e.g., 2 doses, 4 doses, 5 doses, 6 doses, 10 doses) at 6 mg/kg of the patient's body weight at intervals of 4 weeks between doses, followed by multiple doses (e.g., 2 doses, 4 doses, 5 doses, 6 doses, 10 doses, 15 doses, 20 doses) at 10 mg/kg of the patient's body weight at intervals of 4 weeks between doses until the treatment is terminated. An optional dose at 1 mg/kg of the patient's body weight at intervals of 4 weeks between doses (e.g., 2 doses, 4 doses, 5 doses) may be administered prior to dosing at 3 mg/kg, if desired. The subject can be an ApoE4 carrier or an ApoE4 non-carrier.

In a further embodiment of the invention, titration of the monoclonal antibody to the patient can be dispensed with if the patient exhibits the appropriate responses without the titration steps. In this event, for example, an ApoE4 carrier can be administered a dose of the monoclonal antibody of 1 mg/kg, or 3 mg/kg, 6 mg/kg, or 10 mg/kg of the patient's body weight of the anti-Aβ antibody, and an ApoE4 non-carrier can be administered a dose of 3 mg/kg, or 6 mg/kg, or 10 mg/kg, 15 mg/kg, 20 mg/kg, 25 mg/kg, or 30 mg/kg of the patient's body weight of the anti-Aβ antibody. A total of 14 doses can be administered about 4 weeks apart over about 52 weeks, optionally continuing to dose about every 4 weeks thereafter, to thereby treat AD with reduced susceptibility of the patient to ARIA.

Managing ARIA During Treatment with an Anti-Aβ Antibody

Despite the methods described above to prevent or reduce the likelihood of occurrence ARIA, in some instances, a patient may develop ARIA (ARIA-E and/or ARIA-H). This disclosure also provides methods of modifying the treatment of such patients. The methods can involve dose suspension, and/or dose modification, and/or termination of treatment with the anti-Aβ antibody.

### (1) Disposition of ARIA-E Cases

Table 7 below provides a disposition plan for ARIA-E cases that may arise during the treatment regimens described above.

TABLE 7
Disposition Plan for ARIA-E Cases

Clinical

| Symptom | ARIA-E Severity on MRI | | |
|---|---|---|---|
| Severity | Mild | Moderate | Severe |

| | | |
|---|---|---|
| Asymptomatic | Continue dosing at | Suspend dosing. |
| | current dose and | Once ARIA-E resolves the subject may resume |
| | schedule | dosing at the same dose. If the subject previously |
| | | had ARIA-E or ARIA-H that required dose |
| | | suspension, the subject will resume at the next |
| | | lower dose. |

| | |
|---|---|
| Mild | Suspend dosing. Once ARIA-E and clinical symptoms resolve, the subject |
| Moderate | may resume dosing at the same dose. If the subject previously had ARIA-E |
| Severe | or ARIA-H that required dose suspension, the subject will resume at the |
| Serious “other | next lower dose. |
| medically | |
| important | |
| event” only¹ | |
| Serious, except | Discontinue dosing |
| for “other | |
| medically | |
| important | |
| event”² | |

¹“Other medically important events” requiring dose suspension include serious adverse events (SAEs) that are not life-threatening (in the opinion of the Investigator), do not require inpatient hospitalization or prolongation of existing hospitalization, and do not result in significant/permanent disability or congenital anomalies/fetal defects, but may (in the opinion of the Investigator) jeopardize the subject or may require intervention to prevent one of the outcomes listed above.
²SAEs requiring permanent discontinuation of study treatment include those that are life-threatening (in the opinion of the Investigator), require inpatient hospitalization or prolongation of existing hospitalization, and/or result in persistent or significant disability/incapacity or a congenital anomaly/birth defect.

The severity of clinical symptoms are defined as follows:

Mild: Symptom(s) barely noticeable to subject or does not make subject uncomfortable; does not influence performance or functioning; prescription drug not ordinarily needed for relief of symptom(s) but may be given because of personality of subject.

Moderate: Symptom(s) of a sufficient severity to make subject uncomfortable; performance of daily activity is influenced; subject is able to continue in study; treatment for symptom(s) may be needed.

Severe: Symptom(s) cause severe discomfort; symptoms cause incapacitation or significant impact on subject's daily life; severity may cause cessation of treatment with study treatment; treatment for symptom(s) may be given and/or subject hospitalized.

The severity of ARIA-E is defined as follows:

Mild ARIA-E: mild Fluid-attenuated inversion recovery (FLAIR) hyper-intensity confined to sulcus and/or cortex or subcortical white matter (with or without gyral swelling and sulcal effacement) which affects an area of less than 5 cm in single greatest dimension. Only a single region of involvement detected.

Moderate ARIA-E: moderate involvement area of FLAIR hyper-intensity measuring 5-10 cm in single greatest dimension, or more than one site of involvement, each measuring less than 10 cm in single greatest dimension.

Severe ARIA-E: severe involvement (area of FLAIR hyper-intensity measuring greater than 10 cm in single greatest dimension), often with significant subcortical white matter and/or sulcal involvement (with associated gyral swelling and sulcal effacement). One or more separate/independent sites of involvement may be noted.)

According to Table 7, patients who develop mild ARIA-E, per MRI read, with no clinical symptoms at any time during treatment with the anti-Aβ antibody (e.g., BIIB037) can continue treatment with the anti-Aβ antibody at their current dose. Patients should have an MRI approximately every 4 weeks until the ARIA-E has resolved per the MRI read. Patients should also have an MMSE at every scheduled visit until the ARIA-E resolves. A health care practitioner may require that the patients discontinue dosing, or continue dosing at a lower dose level, based on review of safety and MRI data.

Patients who develop moderate or severe ARIA-E, per MRI read, with no clinical symptoms at any time during treatment with the anti-Aβ antibody should temporarily suspend treatment, but should complete all scheduled clinic visits for assessments and, in addition, have an unscheduled visit for an MRI approximately every 4 weeks until the ARIA-E has resolved per MRI. These patients should also have an MMSE at every scheduled visit until the ARIA-E resolves. If the ARIA-E has resolved and the subject remains asymptomatic, the patient may resume treatment at the same dose of the anti-Aβ antibody. If the patient previously had ARIA-E or ARIA-H that required dose suspension, the patient should resume at the next lower dose of the anti-Aβ antibody.

Patients who develop mild, moderate, or severe ARIA-E, per MRI read, accompanied by mild, moderate, severe, or serious (“other medically important event” only) clinical symptoms at any time during treatment with the anti-Aβ antibody should temporarily suspend treatment, but should complete all schedule clinic visits for assessments and, in addition, have an unscheduled visit for an MRI approximately every 4 weeks until the ARIA-E has resolved per the MRI. Patients should also have an MMSE at every scheduled visit until the ARIA-E resolves. If the ARIA-E has resolved and the clinical symptoms have resolved, the patient may resume treatment at the same dose of the anti-Aβ antibody. If the patient previously had ARIA-E or ARIA-H that required dose suspension, the patient will resume at the next lower dose of the anti-Aβ antibody.

Patients who develop mild, moderate, or severe ARIA-E, per MRI read, accompanied by serious (except “other medically important event”) clinical symptoms at any time during the treatment with the anti-Aβ antibody should discontinue treatment with the anti-Aβ antibody. Patients should complete all scheduled clinic visits for assessments and in addition, have an unscheduled visit for an MRI approximately every 4 weeks until the ARIA-E has resolved per centrally read MRI. Patients will also have an MMSE at every scheduled visit until the ARIA-E resolves.

If a patient has a third episode of ARIA that requires dose suspension, the patient discontinues treatment with the anti-Aβ antibody.

### (2) Disposition of ARIA-H (Microhemorrhage) Cases

Table 8 below provides a disposition plan for ARIA-H (microhemorrhage) cases that may arise during the treatment regimens described above.

TABLE 8
Disposition Plan for ARIA-H (Microhemorrhage) Cases

Clinical

| Symptom | Cumulative Microhemorrhages¹ | | |
|---|---|---|---|
| Severity | 1-4 | 5-9 | ≥10 |
| Asymptomatic | Continue dosing at | Suspend dosing. Once | Discontinue dosing |
| | current dose and | ARIA-H is stable the | |
| | schedule | subject may resume dosing | |
| | | at the same dose. If the | |
| | | subject previously had | |
| | | ARIA-E or ARIA-H that | |
| | | required dose suspension, | |
| | | the subject will resume at | |
| | | the next lower dose. | |

| | |
|---|---|
| Mild | Suspend dosing. Once ARIA-H is stable and |
| Moderate | clinical symptoms resolve, the subject may resume |
| Severe | dosing at same dose. If the subject previously had |
| Serious “other | ARIA-E or ARIA-H that required dose suspension, |
| medically | the subject will resume at the next lower dose. |
| important | |
| event” only² | |
| Serious, | Discontinue dosing |
| except for | |
| “other | |
| medically | |
| important | |
| event”³ | |

¹Cumulative microhemorrhages = cumulative microhemorrhages on treatment; does not include microhemorrhages at baseline.
²“Other medically important events” requiring dose suspension include SAEs that are not life-threatening (in the opinion of the Investigator), do not require inpatient hospitalization or prolongation of existing hospitalization, and do not result in significant/permanent disability or congenital anomalies/fetal defects, but may (in the opinion of the Investigator) jeopardize the subject or may require intervention to prevent one of the outcomes listed above.
³SAEs requiring permanent discontinuation of study treatment include those that are life-threatening (in the opinion of the Investigator), require inpatient hospitalization or prolongation of existing hospitalization, and/or result in persistent or significant disability/incapacity or a congenital anomaly/birth defect.

The severity of clinical symptoms are defined as follows:

Mild: Symptom(s) barely noticeable to subject or does not make subject uncomfortable; does not influence performance or functioning; prescription drug not ordinarily needed for relief of symptom(s) but may be given because of personality of subject.

Moderate: Symptom(s) of a sufficient severity to make subject uncomfortable; performance of daily activity is influenced; subject is able to continue in study; treatment for symptom(s) may be needed.

Severe: Symptom(s) cause severe discomfort; symptoms cause incapacitation or significant impact on subject's daily life; severity may cause cessation of treatment with study treatment; treatment for symptom(s) may be given and/or subject hospitalized.

The severity of ARIA-H (microhemorrhage) is defined as follows:

Mild: 1-4 microhemorrhages

Moderate: 5-9 microhemorrhages

Severe: ≥10 microhemorrhages

Patients who develop ≥1 and ≤4 cumulative microhemorrhage(s) with no clinical symptoms during treatment with the anti-Aβ antibody may continue treatment at the current dose, but must have an unscheduled visit for an MRI approximately every 2 weeks until the microhemorrhage is confirmed stable per MRI. The microhemorrhage is considered stable if it is unchanged between 2 consecutive MRIs including the initial detection MRI and the MRI performed 2 weeks later. Patients should also have an MMSE at every scheduled visit until the ARIA-H is stable.

Patients who develop ≥5 and ≤9 cumulative microhemorrhages with no clinical symptoms during treatment with the anti-Aβ antibody should temporarily suspend treatment, but should complete all scheduled clinic visits for assessments and, in addition, have an unscheduled visit for an MRI approximately every 2 weeks until the microhemorrhage is confirmed stable per the MRI. A microhemorrhage is considered stable if it is unchanged between 2 consecutive MRIs including the initial detection MRI and the MRI performed 2 weeks later. Patients will also have an MMSE at every scheduled visit until the ARIA-H is stable. Once the microhemorrhage is deemed stable, patients may resume treatment at the same dose. If the subject previously had ARIA-E or ARIA-H that required dose suspension, the subject will resume at the next lower dose.

Patients who develop ≤9 cumulative microhemorrhage(s) and mild, moderate, severe, or serious (“other medically important event”) clinical symptoms should temporarily suspend treatment with the anti-Aβ antibody, but should complete all scheduled clinic visits for assessments and, in addition, have an unscheduled visit for an MRI approximately every 2 weeks until the microhemorrhage(s) is confirmed stable per MRI. A microhemorrhage is considered stable if it is unchanged between 2 consecutive MRIs including the initial detection MRI and the MRI performed 2 weeks later. Patients should also have an MMSE at every scheduled visit until the ARIA-H is stable. Once the microhemorrhage(s) is deemed stable and the clinical symptoms have resolved, the patient may resume treatment at the same dose of the anti-Aβ antibody. If the subject previously had ARIA-E or ARIA-H that required dose suspension, the patient will resume at the next lower dose of the anti-Aβ antibody.

Patients who experience serious (except “other medically important event”) clinical symptoms associated with microhemorrhage(s) should discontinue treatment, but should complete all scheduled clinic visits for assessments and in addition, have an unscheduled visit for an MRI approximately every 2 weeks until the microhemorrhage(s) is confirmed stable per MRI. Patients will also have an MMSE at every scheduled visit until the ARIA-H is stable.

Patients who develop ≥10 cumulative microhemorrhages, regardless of symptom severity, during treatment with the anti-Aβ antibody should discontinue treatment. Patients should complete all scheduled clinic visits for assessments and in addition, have an unscheduled visit for an MRI approximately every 2 weeks until the microhemorrhages are deemed stable per MRI. Patients will also have an MMSE at every scheduled visit until the ARIA-H is stable.

If a patient has a third episode of ARIA that requires dose suspension, the subject discontinues treatment.

### (3) Disposition of ARIA-H (Superficial Siderosis) Cases

Table 9 below provides a disposition plan for ARIA-H (superficial siderosis) cases that may arise during the treatment regimens described above.

The severity of clinical symptoms are defined as follows:

Mild: Symptom(s) barely noticeable to subject or does not make subject uncomfortable; does not influence performance or functioning; prescription drug not ordinarily needed for relief of symptom(s) but may be given because of personality of subject.

Moderate: Symptom(s) of a sufficient severity to make subject uncomfortable; performance of daily activity is influenced; subject is able to continue in study; treatment for symptom(s) may be needed.

Severe: Symptom(s) cause severe discomfort; symptoms cause incapacitation or significant impact on subject's daily life; severity may cause cessation of treatment with study treatment; treatment for symptom(s) may be given and/or subject hospitalized.

The severity of ARIA-H (superficial siderosis) is defined as follows:

Mild Area of superficial siderosis: 1 new focal region

Moderate Area of superficial siderosis: 2 new focal regions

Severe Area of superficial siderosis: >2 new focal regions.

TABLE 9
Disposition Plan for ARIA-H (Superficial Siderosis) Cases

| Clinical | | | |
|---|---|---|---|
| Symptom | Cumulative Areas of Superficial Siderosis¹ | | |
| Severity | 1 | 2 | >2 |
| Asymptomatic | Continue dosing at | Suspend dosing. Once | Discontinue dosing |
| | current dose and | ARIA-H is stable the subject | |
| | schedule | may resume dosing at the | |
| | | same dose. If the subject | |
| | | previously had ARIA-E or | |
| | | ARIA-H that required dose | |
| | | suspension, the subject will | |
| | | resume at the next lower | |
| | | dose. | |

| | |
|---|---|
| Mild | Suspend dosing. Once ARIA-H is stable and clinical |
| Moderate | symptoms resolve, the subject may resume dosing at |
| Severe | the same dose. If the subject previously had ARIA-E |
| Serious “other | or ARIA-H that required dose suspension, the subject |
| medically | will resume at the next lower dose. |
| important | |
| event” only² | |
| Serious, | Discontinue dosing |
| except for | |
| “other | |
| medically | |
| important | |
| event”³ | |

¹Cumulative superficial siderosis = cumulative superficial siderosis on treatment.
²“Other medically important events” requiring dose suspension include SAEs that are not life-threatening (in the opinion of the Investigator), do not require inpatient hospitalization or prolongation of existing hospitalization, and do not result in significant/permanent disability or congenital anomalies/fetal defects, but may (in the opinion of the Investigator) jeopardize the subject or may require intervention to prevent one of the outcomes listed above.
³SAEs requiring permanent discontinuation of study treatment include those that are life-threatening (in the opinion of the Investigator), require inpatient hospitalization or prolongation of existing hospitalization, and/or result in persistent or significant disability/incapacity or a congenital anomaly/birth defect.

Patients who develop a single focal area of superficial siderosis with no clinical symptoms may continue treatment with the anti-Aβ antibody at the current dose, but must have an unscheduled visit for an MRI approximately every 2 weeks until the superficial siderosis is confirmed stable per the centrally read MRI. Superficial siderosis is considered stable if it is unchanged between 2 consecutive MRIs including the initial detection MRI and the MRI performed 2 weeks later. Patients will also have an MMSE at every scheduled visit until the ARIA-H is stable.

Patients who develop 2 cumulative focal areas of superficial siderosis occurring during treatment with the anti-Aβ antibody with no clinical symptoms should temporarily suspend treatment but should complete all scheduled clinic visits for assessments and, in addition, have an unscheduled visit for an MRI approximately every 2 weeks until the superficial siderosis is confirmed stable per MRI. Superficial siderosis is considered stable if it is unchanged between 2 consecutive MRIs including the initial detection MRI and the MRI performed 2 weeks later. Patients should also have an MMSE at every scheduled visit until the ARIA-H is stable. Once the superficial siderosis is deemed stable, the patient may resume treatment at the same dose. If the patient previously had ARIA-E or ARIA-H that required dose suspension, the subject will resume at the next lower dose.

Patients who develop ≤2 cumulative focal areas of superficial siderosis and mild, moderate, severe, or serious (“other medically important event” only) clinical symptoms should temporarily suspend treatment with the anti-Aβ antibody, but should complete all scheduled clinic visits for assessments and, in addition, have an unscheduled visit for an MRI approximately every 2 weeks until the superficial siderosis is confirmed stable per the centrally read MRI. Superficial siderosis is considered stable if it is unchanged between 2 consecutive MRIs including the initial detection MRI and the MRI performed 2 weeks later. Patients will also have an MMSE at every scheduled visit until the ARIA-H is stable. Once the superficial siderosis is deemed stable and the clinical symptoms have resolved, the patient may resume treatment at the same dose. If the patient previously had ARIA-E or ARIA-H that required dose suspension, the patient should resume at the next lower dose of the anti-Aβ antibody.

Patients who experience serious (except “other medically important event”) clinical symptoms associated with superficial siderosis will discontinue treatment with the anti-Aβ antibody, but should complete all scheduled clinic visits for assessments and in addition, have an unscheduled visit for an MRI approximately every 2 weeks until the superficial siderosis is confirmed stable per MRI. Patients should also have an MMSE at every scheduled visit until the ARIA-H is stable.

Patients who develop >2 cumulative focal areas of superficial siderosis, regardless of clinical symptom severity, should discontinue treatment with the anti-Aβ antibody, but should complete all scheduled clinic visits for assessments and, in addition, have an unscheduled visit for an MRI approximately every 2 weeks until the superficial siderosis is confirmed stable per centrally read MRI. Patients should also have an MMSE at every scheduled visit until the ARIA-H is stable.

If a patient has a third episode of ARIA that requires dose suspension, the patient discontinues treatment.

(4) Disposition of Cases where ARIA-H is Coincident with ARIA-E

Patients who develop ARIA-H coincident with ARIA-E at any time during treatment with the anti-Aβ antibody should follow the most restrictive guidelines of the guidelines discussed above. Prior to resuming treatment, where applicable, ARIA-E must resolve, ARIA-H must be deemed stable, and the subject must be asymptomatic.

### (5) Disposition of ARIA-H (Macrohemorrhage) Cases

Patients who develop any incident macrohemorrhage, regardless of symptom severity during the study, must discontinue treatment with the anti-Aβ antibody, but should complete all scheduled clinic visits for assessments and, in addition, have an unscheduled visit for MRI approximately every 2 weeks until the macrohemorrhage is confirmed stable per MRI. Patients should also have an MMSE at every scheduled visit until the macrohemorrhage is stable.

The severity of ARIA-H (Macrohemorrhage) is defined as follows:

Mild: 1-2 cm in the greatest diameter

Moderate: 2-4 cm in the greatest diameter

Severe: >4 cm in the greatest diameter

### (6) Exemplary Methods of Treating Patients Who Develop ARIA on Standard Dose Regimens

In instances where a patient who is on a standard dose of the anti-Aβ antibody develops moderate or severe ARIA-E with no clinical symptoms a dose suspension is required until the ARIA-E resolves. Once the ARIA-E is resolved, the patient can be administered the same dose that she/he was being administered immediately before development of the moderate or severe ARIA-E. If this patient previously had ARIA-E or ARIA-H that required dose suspension, the patient should be administered a lower dose of the anti-Aβ antibody than that she/he was being administered immediately before development of the most recent moderate or severe ARIA-E. For example, if a patient on a standard dose of 6 mg/kg of the anti-Aβ antibody develops moderate or severe ARIA-E with no clinical symptoms, treatment of the patient with the anti-Aβ antibody should be suspended until the ARIA-E resolves, after which the patient can continue treatment with 6 mg/kg of the anti-Aβ antibody. If, however, this patient had previously developed ARIA-E or ARIA-H that required dose suspension, once the ARIA resolves, the patient should be administered a lower dose (e.g., 1 mg/kg, 2 mg/kg, 3 mg/kg, 4 mg/kg, 5 mg/kg) of the anti-Aβ antibody.

In instances where a patient who is on a standard dose of the anti-Aβ antibody develops mild, moderate, or severe ARIA-E with mild, moderate, severe, or serious clinical symptoms a dose suspension is required until the ARIA-E resolves. Once the ARIA-E is resolved and clinical symptoms resolve, the patient can be administered the same dose that she/he was being administered immediately before development of the moderate or severe ARIA-E. If this patient previously had ARIA-E or ARIA-H that required dose suspension, the patient should be administered a lower dose of the anti-Aβ antibody than that she/he was being administered immediately before development of the most recent moderate or severe ARIA-E with mild, moderate, severe, or serious clinical symptoms. For example, if a patient on a standard dose of 6 mg/kg of the anti-Aβ antibody develops mild, moderate, or severe ARIA-E with mild, moderate, severe, or serious clinical symptoms, treatment of the patient with the anti-Aβ antibody should be suspended until the ARIA-E resolves and clinical symptoms resolve, after which the patient can continue treatment with 6 mg/kg of the anti-Aβ antibody. If, however, this patient had previously developed ARIA-E or ARIA-H that required dose suspension, once the ARIA resolves and clinical symptoms resolve, the patient should be administered a lower dose (e.g., 1 mg/kg, 2 mg/kg, 3 mg/kg, 4 mg/kg, 5 mg/kg) of the anti-Aβ antibody.

In instances where a patient who is on a standard dose of the anti-Aβ antibody develops 5 to 9 cumulative microhemorrhages with no clinical symptoms a dose suspension is required until the ARIA-H is stable. Once the ARIA-H is stable, the patient can be administered the same dose that she/he was being administered immediately before development of 5 to 9 cumulative microhemorrhages. If this patient previously had ARIA-E or ARIA-H that required dose suspension, the patient should be administered a lower dose of the anti-Aβ antibody than that she/he was being administered immediately before development of the 5 to 9 cumulative microhemorrhages. For example, if a patient on a standard dose of 6 mg/kg of the anti-Aβ antibody develops 5 to 9 cumulative microhemorrhages with no clinical symptoms, treatment of the patient with the anti-Aβ antibody should be suspended until the ARIA-H is stable, after which the patient can continue treatment with 6 mg/kg of the anti-Aβ antibody. If, however, this patient had previously developed ARIA-E or ARIA-H that required dose suspension, once the ARIA-H stabilizes, the patient should be administered a lower dose (e.g., 1 mg/kg, 2 mg/kg, 3 mg/kg, 4 mg/kg, 5 mg/kg) of the anti-Aβ antibody.

In instances where a patient who is on a standard dose of the anti-Aβ antibody develops 1 to 9 cumulative microhemorrhages with mild, moderate, severe, or serious clinical symptoms a dose suspension is required until the ARIA-H is stable. Once the ARIA-H is stable and clinical symptoms resolve, the patient can be administered the same dose that she/he was being administered immediately before development of 1 to 9 cumulative microhemorrhages. If this patient previously had ARIA-E or ARIA-H that required dose suspension, the patient should be administered a lower dose of the anti-Aβ antibody than that she/he was being administered immediately before development of the 1 to 9 cumulative microhemorrhages with mild, moderate, severe, or serious clinical symptoms. For example, if a patient on a standard dose of 6 mg/kg of the anti-Aβ antibody develops 1 to 9 cumulative microhemorrhages with mild, moderate, severe, or serious clinical symptoms, treatment of the patient with the anti-Aβ antibody should be suspended until the ARIA-H is stable and clinical symptoms resolve, after which the patient can continue treatment with 6 mg/kg of the anti-Aβ antibody. If, however, this patient had previously developed ARIA-E or ARIA-H that required dose suspension, once the ARIA-H stabilizes and clinical symptoms resolve, the patient should be administered a lower dose (e.g., 1 mg/kg, 2 mg/kg, 3 mg/kg, 4 mg/kg, 5 mg/kg) of the anti-Aβ antibody.

In instances where a patient who is on a standard dose of the anti-Aβ antibody develops 2 cumulative areas of superficial siderosis with no clinical symptoms a dose suspension is required until the ARIA-H is stable. Once the ARIA-H is stable, the patient can be administered the same dose that she/he was being administered immediately before development of 2 cumulative areas of superficial siderosis. If this patient previously had ARIA-E or ARIA-H that required dose suspension, the patient should be administered a lower dose of the anti-Aβ antibody than that she/he was being administered immediately before development of the 2 cumulative areas of superficial siderosis. For example, if a patient on a standard dose of 6 mg/kg of the anti-Aβ antibody develops 2 cumulative areas of superficial siderosis with no clinical symptoms, treatment of the patient with the anti-Aβ antibody should be suspended until the ARIA-H is stable, after which the patient can continue treatment with 6 mg/kg of the anti-Aβ antibody. If, however, this patient had previously developed ARIA-E or ARIA-H that required dose suspension, once the ARIA-H stabilizes, the patient should be administered a lower dose (e.g., 1 mg/kg, 2 mg/kg, 3 mg/kg, 4 mg/kg, 5 mg/kg) of the anti-Aβ antibody.

Where a patient who is on a standard dose of the anti-Aβ antibody develops 1 or 2 cumulative areas of superficial siderosis with mild, moderate, severe, or serious clinical symptoms a dose suspension is required until the ARIA-H is stable and clinical symptoms resolve. Once the ARIA-H is stable and clinical symptoms resolve, the patient can be administered the same dose that she/he was being administered immediately before development of 2 cumulative areas of superficial siderosis. If this patient previously had ARIA-E or ARIA-H that required dose suspension, the patient should be administered a lower dose of the anti-Aβ antibody than that she/he was being administered immediately before development of the 1 or 2 cumulative areas of superficial siderosis. For example, if a patient on a standard dose of 6 mg/kg of the anti-Aβ antibody develops 1 or 2 cumulative areas of superficial siderosis with mild, moderate, severe, or serious clinical symptoms, treatment of the patient with the anti-Aβ antibody should be suspended until the ARIA-H is stable and clinical symptoms resolve, after which the patient can continue treatment with 6 mg/kg of the anti-Aβ antibody. If, however, this patient had previously developed ARIA-E or ARIA-H that required dose suspension, once the ARIA-H stabilizes and clinical symptoms resolve, the patient should be administered a lower dose (e.g., 1 mg/kg, 2 mg/kg, 3 mg/kg, 4 mg/kg, 5 mg/kg) of the anti-Aβ antibody.

### (7) Exemplary Methods of Treating Patients Who Develop ARIA on Titration Regimens

In instances where a patient who is on a titration regimen of the anti-Aβ antibody develops moderate or severe ARIA-E with no clinical symptoms a dose suspension is required until the ARIA-E resolves. Once the ARIA-E is resolved, the patient can be administered the same dose that she/he was being administered immediately before development of the moderate or severe ARIA-E. If this patient previously had ARIA-E or ARIA-H that required dose suspension, the patient should be administered a lower dose of the anti-Aβ antibody than that she/he was being administered immediately before development of the most recent ARIA requiring dose suspension. For example, if the patient is on the Protocol (5) regimen described above and develops moderate or severe ARIA-E with no clinical symptoms after step (C), treatment with the anti-Aβ antibody should be suspended until the ARIA-E resolves. Once the ARIA-E is resolved, the patient can be administered the same dose that she/he was being administered immediately before development of the moderate or severe ARIA-E (i.e., 3 mg/kg of body weight of the patient). When treatment with the anti-Aβ antibody resumes after the dose suspension, patients must have a minimum of 2 doses at the restarted dose (i.e., at least 2 doses of 3 mg/kg). An MRI should be performed after the second administration of the restarted dose, and after the second administration of each increase in dose. After that, the patient can continue with the remaining steps of Protocol (5) (i.e., Steps (D) through (L)).

If, however, the patient being treated according to the Protocol (5) regimen who develops moderate or severe ARIA-E with no clinical symptoms after step (C), previously had ARIA-E or ARIA-H that required dose suspension, treatment with the anti-Aβ antibody should be suspended until the ARIA-E resolves, and once the ARIA-E resolves, this patient should be administered a lower dose of the anti-Aβ antibody than that she/he was being administered immediately before development of the most recent moderate or severe ARIA requiring dose suspension (in this case 1 mg/kg of body weight of the patient). When treatment with the anti-Aβ antibody resumes after the dose suspension, patients must have a minimum of 2 doses at the restarted dose (i.e., at least 2 doses of 1 mg/kg). An MRI should be performed after the second administration of the restarted dose, and after the second administration of each increase in dose. After that, the patient can continue with the remaining steps of Protocol (5) (i.e., Steps (D) through (L)).

In instances where a patient who is on a titration regimen of the anti-Aβ antibody develops mild, moderate, or severe ARIA-E with mild, moderate, or severe clinical symptoms, or clinical symptoms meeting the “other medically important” serious criteria a dose suspension is required until the ARIA-E resolves. Once the ARIA-E is resolved and clinical symptoms resolve, the patient can be administered the same dose that she/he was being administered immediately before development of the mild, moderate or severe ARIA-E. If this patient previously had ARIA-E or ARIA-H that required dose suspension, the patient should be administered a lower dose of the anti-Aβ antibody than that she/he was being administered immediately before development of the most recent ARIA requiring dose suspension. For example, if the patient is on the Protocol (5) regimen described above and develops mild, moderate or severe ARIA-E with mild, moderate, or severe clinical symptoms, or clinical symptoms meeting the “other medically important” serious criteria after step (E), treatment with the anti-Aβ antibody should be suspended until the ARIA-E resolves and the clinical symptoms resolve. Once the ARIA-E and the clinical symptoms resolve, the patient can be administered the same dose that she/he was being administered immediately before development of the moderate or severe ARIA-E (i.e., 3 mg/kg of body weight of the patient). When treatment with the anti-Aβ antibody resumes after the dose suspension, patients must have a minimum of 2 doses at the restarted dose (i.e., at least 2 doses of 3 mg/kg). An MRI should be performed after the second administration of the restarted dose, and after the second administration of each increase in dose. After that, the patient can continue with the remaining steps of Protocol (5) (i.e., Steps (F) through (L)).

If, however, the patient being treated according to the Protocol (5) regimen who develops mild, moderate, or severe ARIA-E with mild, moderate, or severe clinical symptoms, or clinical symptoms meeting the “other medically important” serious criteria after step (E), previously had ARIA-E or ARIA-H that required dose suspension, treatment with the anti-Aβ antibody should be suspended until the ARIA-E resolves and/or ARIA-H is stable and the clinical symptoms resolve, and once they resolve, this patient should be administered a lower dose of the anti-Aβ antibody than that she/he was being administered immediately before development of the most ARIA (in this case 1 mg/kg of body weight of the patient). When treatment with the anti-Aβ antibody resumes after the dose suspension, patients must have a minimum of 2 doses at the restarted dose (i.e., at least 2 doses of 1 mg/kg). An MRI should be performed after the second administration of the restarted dose, and after the second administration of each increase in dose. After that, the patient can continue with the remaining steps of Protocol (5) (i.e., Steps (F) through (L)).

If, for example, the patient is on the Protocol (5) regimen described above and develops mild, moderate or severe ARIA-E with mild, moderate, severe, or clinical symptoms meeting the “other medically important” serious criteria after step (G), treatment with the anti-Aβ antibody should be suspended until the ARIA-E resolves and the clinical symptoms resolve. Once the ARIA-E and the clinical symptoms resolve, the patient can be administered the same dose that she/he was being administered immediately before development of the moderate or severe ARIA-E (i.e., 6 mg/kg of body weight of the patient). When treatment with the anti-Aβ antibody resumes after the dose suspension, patients must have a minimum of 2 doses at the restarted dose (i.e., at least 2 doses of 6 mg/kg). An MRI should be performed after the second administration of the restarted dose, and after the second administration of each increase in dose. After that, the patient can continue with the remaining steps of Protocol (5) (i.e., Steps (H) through (L)). If, however, the patient being treated according to the Protocol (5) regimen who develops mild, moderate, or severe ARIA-E with mild, moderate, severe, or serious clinical symptoms after step (G), previously had ARIA-E or ARIA-H that required dose suspension, treatment with the anti-Aβ antibody should be suspended until the ARIA-E resolves and the clinical symptoms resolve, and once they resolve, this patient should be administered a lower dose of the anti-Aβ antibody than that she/he was being administered immediately before development of the most recent moderate or severe ARIA-E (in this case 3 mg/kg of body weight of the patient). When treatment with the anti-Aβ antibody resumes after the dose suspension, patients must have a minimum of 2 doses at the restarted dose (i.e., at least 2 doses of 3 mg/kg). An MRI should be performed after the second administration of the restarted dose, and after the second administration of each increase in dose. After that, the patient can continue with the remaining steps of Protocol (5) (i.e., Steps (F) through (L)).

In instances where a patient who is on a titration regimen of the anti-Aβ antibody develops 5 to 9 cumulative microhemorrhages with no clinical symptoms a dose suspension is required until the ARIA-H is stable. Once the ARIA-H is stable, the patient can be administered the same dose that she/he was being administered immediately before development of 5 to 9 cumulative microhemorrhages. If this patient previously had ARIA-E or ARIA-H that required dose suspension, the patient should be administered a lower dose of the anti-Aβ antibody than that she/he was being administered immediately before development of the 5 to 9 cumulative microhemorrhages. For example, if a patient on the Protocol (5) treatment regimen of the anti-Aβ antibody develops 5 to 9 cumulative microhemorrhages with no clinical symptoms after step (D), treatment of the patient with the anti-Aβ antibody should be suspended until the ARIA-H is stable, after which the patient can continue treatment with the same amount of the anti-Aβ antibody of step (D) (i.e., 3 mg/kg of body weight of the patient). When treatment with the anti-Aβ antibody resumes after the dose suspension, patients must have a minimum of 2 doses at the restarted dose (i.e., at least 2 doses of 3 mg/kg). After that, the patient can continue with the remaining steps of Protocol (5) (i.e., Steps (E) through (L)).

If, however, this patient had previously developed ARIA-E or ARIA-H that required dose suspension, once the ARIA-H stabilizes, the patient should be administered a lower dose of Protocol (5) (i.e., 1 mg/kg of the body weight of the patient) of the anti-Aβ antibody. The patient is administered a minimum of 2 doses of 1 mg/kg of the body weight of the patient of the anti-Aβ antibody. After that, the patient may continue with the remaining steps of Protocol (5) (i.e., Steps (E) through (L)).

In instances where a patient who is on a titration regimen of the anti-Aβ antibody develops 1 to 9 cumulative microhemorrhages with mild, moderate, severe, or serious clinical symptoms a dose suspension is required until the ARIA-H is stable and the clinical symptoms resolve. Once the ARIA-H is stable and the clinical symptoms resolve, the patient can be administered the same dose that she/he was being administered immediately before development of 1 to 9 cumulative microhemorrhages. If this patient previously had ARIA-E or ARIA-H that required dose suspension, the patient should be administered a lower dose of the anti-Aβ antibody than that she/he was being administered immediately before development of the 1 to 9 cumulative microhemorrhages with mild, moderate, or severe clinical symptoms, or clinical symptoms meeting the “other medically important” serious criteria. For example, if a patient on Protocol (5) develops 1 to 9 cumulative microhemorrhages with mild, moderate, severe, or serious clinical symptoms, after step (E), treatment of the patient with the anti-Aβ antibody should be suspended until the ARIA-H is stable and clinical symptoms resolve, after which the patient can continue treatment with the same amount of the anti-Aβ antibody as used in step (E) (i.e., 3 mg/kg of body weight of the patient). When treatment with the anti-Aβ antibody resumes after the dose suspension, patients must have a minimum of 2 doses at the restarted dose (i.e., at least 2 doses of 3 mg/kg). After that, the patient can continue with the remaining steps of Protocol (5) (i.e., Steps (F) through (L)).

If, however, this patient had previously developed ARIA-E or ARIA-H that required dose suspension, once the ARIA-H stabilizes and clinical symptoms resolve, the patient should be administered a lower dose (i.e., 1 mg/kg of body weight of the patient) of the anti-Aβ antibody. The patient is administered a minimum of 2 doses of 1 mg/kg of the body weight of the patient of the anti-Aβ antibody. After that, the patient may continue with the remaining steps of Protocol (5) (i.e., Steps (F) through (L)).

In instances where a patient who is on a titration regimen of the anti-Aβ antibody develops 2 cumulative areas of superficial siderosis with no clinical symptoms a dose suspension is required until the ARIA-H is stable. Once the ARIA-H is stable, the patient can be administered the same dose that she/he was being administered immediately before development of 2 cumulative areas of superficial siderosis. If this patient previously had ARIA-E or ARIA-H that required dose suspension, the patient should be administered a lower dose of the anti-Aβ antibody than that she/he was being administered immediately before development of the 2 cumulative areas of superficial siderosis. For example, if a patient on Protocol (5) develops 2 cumulative areas of superficial siderosis with no clinical symptoms after step (E), treatment of the patient with the anti-Aβ antibody should be suspended until the ARIA-H is stable, after which the patient can continue treatment with the same amount of the antibody as in step (E) (i.e., 3 mg/kg of body weight of the patient). The patient is administered a minimum of 2 doses of 3 mg/kg of the body weight of the patient of the anti-Aβ antibody. After that, the patient can continue with the remaining steps of Protocol (5) (i.e., Steps (F) through (L)).

If, however, this patient had previously developed ARIA-E or ARIA-H that required dose suspension, once the ARIA-H stabilizes, the patient should be administered a minimum of 2 doses of the next lower dose of the Protocol (i.e., 1 mg/kg of body weight of the patient) of the anti-Aβ antibody. After that, the patient may continue with the remaining steps of Protocol (5) (i.e., Steps (F) through (L)).

Where a patient who is on a titration regimen of the anti-Aβ antibody develops 1 or 2 cumulative areas of superficial siderosis with mild, moderate, or severe clinical symptoms, or clinical symptoms meeting the “other medically important” serious criteria a dose suspension is required until the ARIA-H is stable and clinical symptoms resolve. Once the ARIA-H is stable and the clinical symptoms resolve, the patient can be administered the same dose that she/he was being administered immediately before development of 2 cumulative areas of superficial siderosis. If this patient previously had ARIA-E or ARIA-H that required dose suspension, the patient should be administered a lower dose of the anti-Aβ antibody than that she/he was being administered immediately before development of the 1 or 2 cumulative areas of superficial siderosis. For example, if a patient on Protocol (5) develops 1 or 2 cumulative areas of superficial siderosis with mild, moderate, severe, or serious clinical symptoms after Step (C), treatment of the patient with the anti-Aβ antibody should be suspended until the ARIA-H is stable and clinical symptoms resolve, after which the patient can continue treatment with a minimum of two doses of the same amount of the anti-Aβ antibody as Step (C) of Protocol (5) (i.e., 3 mg/kg of the body weight of the patient). After that, the patient can continue with the remaining steps of Protocol (5) (i.e., Steps (D) through (L)).

If, however, this patient had previously developed ARIA-E or ARIA-H that required dose suspension, once the ARIA-H stabilizes and clinical symptoms resolve, the patient should be administered a minimum of two doses of the next lower amount of the anti-Aβ antibody of Protocol (5) (i.e., 1 mg/kg of the body weight of the patient). After that, the patient may continue with the remaining steps of Protocol (5) (i.e., Steps (D) through (L)).

(8) Restarting Treatment after Dose Suspension Due to ARIA

In all of the above cases, when treatment with an anti-Aβ antibody (e.g., BIIB037) resumes after a dose suspension, patients must have a minimum of 2 doses at the restarted dose. An MRI should be performed after the second administration of the restarted dose, and after the second administration of each increase in dose.

### Measurement and Reduction in the Symptoms of AD

Measurement of the risk, existence, severity, and progression of AD can be determined by clinical diagnosis over time; assessment of the global functional level of the patient; evaluation of the daily living capacities or behavioral deficits; volumetric analysis of brain structures; in vivo measurement of pathological deposits of abnormal proteins in brain (e.g. PET beta-amyloid imaging), or biochemical variables in body fluids (e.g. tau proteins or Aβ peptides); and by comparison to the natural course/history of the disease.

The following clinical assessments can be employed in determining the stage of AD in the patient: CDR, FCSRT, Neuropsychiatric Inventory-Questionnaire (NPI-Q), and a neuropsychological test battery comprising Rey Auditory Verbal Learning Test (RA VLT) Immediate and Delayed Recall, Wechsler Memory Scale (WMS) Verbal Pair Associate Learning Test Immediate and Delayed Recall, Delis-Kaplan Executive Function System Verbal Fluency Conditions 1 and 2, and the Wechsler Adult Intelligence Scale Fourth Edition Symbol Search and Coding Subsets; and the Cognitive Drug Research computerized test battery.

In one embodiment, a diagnostic regime comprises determining the change from baseline on the Clinical Dementia Rating (CDR) Scale, a neuropsychological test battery, Cognitive Drug Research computerized test battery, the Free and Cued Selective Reminding Test (FCSRT), Mini Mental State Examination (MMSE), Columbia Suicide Severity Rating Scale (C-SSRS), and Neuropsychiatric Inventory-Questionnaire (NPI-Q).

Biomarkers have emerged as essential for defining AD and for staging of the disease along its spectrum. Biomarker phenotypes can bridge the gap between clinical phenotypes and neuropathology phenotypes, such as amyloid plaques, neurofibrillary tangles, inflammation, and neurodegeneration. Biomarkers of AD include ApoE isotype, CSF Aβ42, amyloid PET, CSF Tau, and hippocampal volumetric (HCV) MRI.

Amyloid plaque burden in certain areas of the brain can be measured by 18F-AV-45 PET. 18F-AV-45 is an amyloid ligand developed by Avid Radiopharmaceuticals (Philadelphia, Pa.). It binds to fibrillar Aβ with a high affinity (Kd=3.1 nM). Results with 18F-AV-45 PET imaging have shown that patients with AD have selective retention of tracer in cortical areas expected to be high in amyloid deposition, whereas healthy controls have shown rapid washout from these areas, with only minimal cortical tracer retention. A significant difference in mean uptake of 18F-AV-45 has been observed between AD and age-matched control subjects. Test-retest variance of 18F-AV-45 PET imaging is low (less than 5%) in both AD patients and cognitively healthy controls. Visual interpretation of the 18F-AV-45 PET images and mean quantitative estimates of cortical uptake correlate with presence and quantity of amyloid pathology at autopsy as measured by immunohistochemistry and silver stain neuritic plaque score (Clark C M, et al. Use of florbetapir-PET for imaging β-amyloid pathology. JAMA, 2011 January; 305(3):275-283).

Radiation dosimetry of 18F-AV-45 is in the range of typical PET ligands. The average human whole body effective dose is estimated to be 0.019 mSv/MBq. A dose of 370 MBq per injection has also been shown to yield good imaging results.

Patients with AD have characteristic reductions in FDG PET measurements of regional glucose metabolism, which are related to progressive impairment of cognitive function (Landau S M, et al. Associations between cognitive, functional, and FDG-PET measures of decline in AD and MCI. Neurobiol Aging, 2011 July; 32(7):1207-18; Mielke R, et al. HMPAO SPET and FDG PET in Alzheimer's disease and vascular dementia: comparison of perfusion and metabolic pattern. Eur J Nucl Med., 1994 October; 21(10):1052-60). The effect of anti-Aβ antibody in halting the progression of glucose metabolic deficit can be periodically assessed using FDG PET measurements. Radiation dosimetry of FDG is in the range of typical PET ligands. The average human whole body effective dose is estimated to be 0.019 mSv/MBq. The standard FDG imaging protocol uses a dose of 185 MBq per injection. Patients can typically receive up to 185 MBq with each scan.

Measurement of Aβ 1-42 and T-Tau or P-Tau levels in CSF are gaining acceptance as predictive biomarkers of AD. Evidence suggests that Tau aggregation pathology is a very early event in pathogenesis. (Duyckaerts (2011) Lancet Neurol. 10, 774-775, and Braak et al., (2013), Acta Neuropath., 126:631-41).

AD-related biomarkers can also be employed. These include, but are not limited, to pyroglutamate-Aβ, Aβ40, and Aβ42 in blood, and total Tau, phospho-Tau, pyroglutamate-Aβ Aβ40, and Aβ42 in CSF.

Morphometric MRI measures can also aid in the assessment of AD. These include whole brain volume, hippocampal volume, ventricle volume, and cortical gray matter volume. Cerebral blood flow as measured by ASL-MRI and functional connectivity as measured by tf-fMRI can be included in the assessment protocols.

Use of an anti-Aβ antibody (e.g., BIIB037) for the treatment of AD patients according to the disclosure results in an improvement in one or more of these parameters over baseline measurements or at least prevents or slows the progression of AD from one stage to the next stage.

### Measurement of ARIA

AD patients generally respond to anti-Aβ antibody (e.g., BIIB037) in a dose dependent manner. Therefore, it is advantageous to use high doses for maximum effectiveness. But the incidence or rate of ARIA can increase in certain patient populations when doses of the anti-Aβ antibody are increased. This disclosure makes it possible to reduce the incidence of ARIA in susceptible patients undergoing treatment for Alzheimer's disease, especially those patients receiving high doses of the anti-Aβ antibody, as well as ApoE4 carriers. In particular, this disclosure makes it possible to reduce the incidence of amyloid related imaging abnormalities-edema (ARIA-E), or reduce the incidence of amyloid related imaging abnormalities-hemorrhage or hemosiderosis (ARIA-H), or reduce both ARIA-E and ARIA-H.

ARIA, including edema (ARIA-E) and microhemorrhage or hemosiderosis (ARIA-H), are readily detectable by MRI (i.e., fluid attenuated inversion recovery (FLAIR/T2 for ARIA-E and T2*/gradient echo for ARIA-H). (Sperling R, et al. Amyloid-related imaging abnormalities in patients with Alzheimer's disease treated with bapineuzumab: a retrospective analysis. Lancet Neurol., 2012; 11(3):241-9). Susceptibility weighted imaging (SWI), an MRI technique potentially more sensitive than T2*/gradient echo in detecting ARIA-H (Sperling R A, et al. Amyloid-related imaging abnormalities in amyloid-modifying therapeutic trials: Recommendations from the Alzheimer's Association Research Roundtable Workgroup. Alzheimer's and Dementia, 2011; 7(4):367-85), can also be employed.

Signs of vasogenic edema include hyperintense signal on T2-weighted and FLAIR sequences generally confined to the white matter and often associated with gyral swelling. Symptoms of vasogenic edema when present include headache, worsening cognitive function, alteration of consciousness, seizures, unsteadiness, and vomiting.

ARIA-H is monitorable by MRI and believed to be an imaging finding without clinical correlate (i.e., patients are asymptomatic) (Sperling R A, et al. Amyloid-related imaging abnormalities in amyloid-modifying therapeutic trials: Recommendations from the Alzheimer's Association Research Roundtable Workgroup. Alzheimer's and Dementia, 2011; 7(4):367-85). Specifically, hemorrhage is detectable using MRI sequences of gradient echo, T1-weighted, T2-weighted, and FLAIR. Microhemorrhage is usually asymptomatic, whereas macrohemorrhage typically has focal signs and symptoms reflecting the area of the affected brain as well as non-specific symptoms that include those for vasogenic edema. The frequency of MRI acquisition is driven by safety monitoring needs.

The following are examples of the practice of the invention. They are not to be construed as limiting the scope of the invention in any way.

### EXAMPLES

### Example 1: Toxicology Study of BIIB037 In Vivo

The Tg2576 mouse and cynomolgus monkey were used for BIIB037 toxicology evaluation. Of the 2 species, the Tg2576 mouse is considered the primary pharmacologically relevant species given that these mice accumulate amyloid plaques in the cerebral parenchyma and vasculature.

In addition to the standard histopathologic evaluation in mice, Perls staining of hemosiderin (a breakdown product of hemoglobin) was performed to quantify microhemorrhage. Microhemorrhage has been observed both as a background finding in transgenic mouse models of AD (Winkler D T, et al. Spontaneous hemorrhagic stroke in a mouse model of cerebral amyloid angiopathy. J. Neurosci., 2001 Mar. 1; 21(5):1619-27), including Tg2576 mice (Kumar-Singh S, et al. Dense-core plaques in Tg2576 and PSAPP mouse models of Alzheimer's disease are centered on vessel walls. American Journal of Pathology, 2005 August; 167(2):527-43), and as a drug-related finding in transgenic mice treated with some anti-Aβ antibodies [Pfeifer M, et al. Cerebral hemorrhage after passive anti-Aβ immunotherapy. Science 2002 Nov. 15; 298(5597):1379; Racke M M, et al. Exacerbation of cerebral amyloid angiopathy-associated microhemorrhage in amyloid precursor protein transgenic mice by immunotherapy is dependent on antibody recognition of deposited forms of amyloid beta. J Neurosci., 2005 Jan. 19; 25(3):629-36.; Wilcock O M, Colton C A. Immunotherapy, vascular pathology, and microhemorrhages in transgenic mice. CNS & Neurological Disorders Drug Targets, 2009 March; 8(1):50-64).

### Example 2: Short Term Study of BIIB037 In Vivo

In a 13-week study, Tg2576 mice were administered weekly IV doses of 10 or 70 mg/kg of ch12F6A, or 500 mg/kg of either ch12F6A or BIIB037. Minimal to mild acute hemorrhage was observed in 2 mice dosed at >70 mg/kg/week as assessed by the standard histopathologic staining Additional findings included a slight increase in the incidence and/or severity of meningeal vascular inflammation in mice treated at >70 mg/kg/week compared with control animals, and the occurrence of thrombosis in 2 animals dosed at 500 mg/kg/week. At the end of a 6-week drug-free recovery period, the incidence and severity of findings observed in ch12F6A and BIIB037-treated mice were within the range observed in the control group throughout the study.

In addition to standard histopathology of the brain, presence of microhemorrhage was evaluated by Perls staining; no significant differences in microhemorrhage were observed between ch12F6A/BIIB037 and control treated groups after 13 weeks of dosing.

The increased incidence and/or severity of meningeal vascular inflammation and acute hemorrhage observed at or greater than 70 mg/kg/week contributed towards the no observed adverse effect level (NOAEL) determination of 10 mg/kg/week.

### Example 3: Longer Term Study of BIIB037 In Vivo

In a 6-month study, Tg2576 mice were administered weekly IV doses of 10 or 40 mg/kg of ch12F6A, or 250 mg/kg of either ch12F6A or BIIB037. There were no treatment-related changes in any of the parameters evaluated during the main and recovery periods, with the exception of a slight increase in the combined incidence and/or severity of meningeal/cerebral vascular inflammation and vascular thickening in the brains of main and early death animals treated with chimeric 12F6A (ch12F6A) comprising murine constant domains at doses >40 mg/kg, and an increase in area of micro hemorrhage in a subset of the 250 mg/kg ch12F6A-treated animals.

There were no treatment-related findings, nor increase in the incidence and/or severity of meningeal/cerebral vascular inflammation and/or vascular thickening in Tg2576 mice that received weekly intravenous injection administration of 250 mg/kg BIIB037 and no statistically significant difference in the number of foci and/or percent area of microhemorrhage in the brain of animals receiving ch12F6A or BIIB037.

After a 6-week recovery period, the incidence and/or severity of the vascular inflammation or thickening was similar across treated and control groups. Although a potential treatment-related exacerbation of these changes cannot be totally excluded, the vascular inflammation, thickening, and possible exacerbated microhemorrhage in the brain were considered of equivocal relationship to treatment and potentially due to the age-related degenerative changes inherent to the disease model alone. Consequently, the NOAEL is 250 mg/kg/week for this study.

No treatment-related findings were observed in a 4-week monkey study, the NOAEL was 300 mg/kg/week.

In summary, the toxicology evaluation for BIIB037 identified a toxicity profile consistent with binding of the antibody to deposited Aβ.

### Example 4: Reduction of Amyloid Beta In Vivo

In Tg2576 mice, a dose-dependent reduction in cerebral amyloid was observed after chronic dosing with ch12F6A (0.3 mg/kg to 30 mg/kg). A significant amyloid reduction was observed at 3 mg/kg, deemed the minimal effective dose, and efficacy appeared to reach a plateau between 10 mg/kg and 30 mg/kg. The no observed adverse effect level (NOAEL) obtained from a 13-week Tg2576 mouse toxicology study (10 mg/kg/week) was used for the purpose of safety margin determination.

BIIB037 mean steady state exposure in humans (calculated as AUC_{0-4 wk}) at 1 and 3 mg/kg is projected to be approximately one-twelfth and one-fourth the nonclinical NOAEL dose exposure (calculated as AUC₀₋₄ wk) observed in the 13-week mouse toxicology study. BIIB037 mean steady state exposure following a 10 mg/kg dose is projected to be similar to NOAEL dose exposures. The highest dose, 30 mg/kg, is projected to achieve mean steady state exposures 2- to 3-times the NOAEL exposure and one-third the exposure at the 70 mg/kg dose where slight increases in the severity of meningeal vascular inflammation and incidences of cerebral hemorrhage were observed.

### Example 5: Clinical Experience with BIIB037

The first clinical study is a Phase 1, randomized, blinded, placebo-controlled single ascending dose (SAD) study of the safety, tolerability, and pharmacokinetics (PK) of BIIB037 in subjects with mild to moderate AD. Fifty-three subjects were enrolled in the SAD study.

The starting dose of BIIB037 was 0.3 mg/kg, increasing to 60 mg/kg, a dose predicted to provide a mean exposure (AUCinf) that does not exceed the mean exposure in Tg2576 mice given 500 mg/kg (AUCTAU=402000 μg*hr/mL). Doses up to 30 mg/kg (0.3, 1, 3, 10, 20, and 30 mg/kg) were generally well tolerated.

Two serious adverse events (SAEs) of symptomatic amyloid related imaging abnormalities-edema (ARIA-E), and one adverse event (AE) of asymptomatic ARIA-E were reported in the 60 mg/kg cohort. Further enrollment into the 60 mg/kg cohort was terminated per study protocol. No deaths or withdrawals due to AEs were reported in the SAD study. Serum exposures of BIIB037 have demonstrated linearity with doses up through 30 mg/kg.

### Example 6: Clinical Study of BIIB037

### A. Phase 1b Clinical Study of BIIB037 in Human AD Subjects

A Phase 1b clinical trial was conducted. The trial was a randomized, blinded, placebo-controlled, ascending dose study of BIIB037 in prodromal to mild AD subjects and positive amyloid scans. The primary endpoint of the trial was safety. Secondary endpoints included assessment of the effect on cerebral amyloid plaque content as measured by 18F-AV-45 PET imaging. Change from baseline in 18F-AV-45 PET signal was assessed in certain brain areas. Exploratory endpoints assessed cognition in the subjects. Subjects received 1, 3, 6, or 10 mg/kg of BIB037 based on the patient's body weight, or placebo.

### B. Pre-Specified Interim Analysis #1

Pre-specified Interim Analysis #1 provided 26 week data for the 1, 3, and 10 mg/kg groups and the placebo group.

The AD subjects were randomized into 4 groups, placebo, those receiving BIIB037 at 1 mg/kg of the patient's body weight, those receiving BIIB037 at 3 mg/kg of body weight, and those receiving BIIB037 at 10 mg/kg of body weight. There were approximately 31 subjects in each group. The average age of the subjects was about 72 years (mean). Apo E4 carriers comprised to 63%, 61%, 66%, and 63%, of the groups, respectively.

The clinical stage of AD in the subjects was assessed. Subjects with prodromal AD comprised to 47%, 32%, 44%, and 41% of the groups, respectively. Subjects with mild AD comprised to 53%, 68%, 56%, and 59% of the groups, respectively.

A static PET acquisition protocol was employed. Tracer was injected into each subject and a single scan was conducted. The tracer was AV45, a PET ligand targeting fibrillar Aβ plaques.

The results of the amyloid PET imaging protocol were expressed as a standard update value ratio, which is a measure of the uptake of the β-amyloid ligand used for PET imaging and corresponds to the amount of β-amyloid present. The standardized uptake value ratio normalizes the PET signal by taking a ratio of a target region over a reference region. In the target region, specific binding and change in binding signal reflect treatment-induced modulation of pharmacology. In the reference region, nonspecific binding indicates no effect of the treatment.

A dose-dependent reduction of amyloid was observed. There was a statistically significant reduction observed at 3 mg/kg and at 10 mg/kg at week 26. The effect appeared to continue to week 54 based on a small subset of subjects. There was no obvious ApoE modification of the observed effects. Greater effects were observed in subjects with higher baseline standard update value ratios.

Safety and tolerability of the treatment were assessed. Adverse events were generally mild or moderate. Headache was the most common adverse event and appeared to be dose-dependent. There were no significant changes in chemistry, hematology, urinalysis, ECGs, or vital signs. Twenty seven subjects exhibited ARIA-E or ARIA-E/H.

Higher incidence of ARIA was observed with higher BIIB037 doses and with Apo E4 carriage. Homozygous and heterozygous E4-carriers appeared to be at a similar risk for ARIA.

The onset of ARIA-E usually occurred early in the course of treatment. ARIA-E occurred at doses of 1 and 3 mg/kg after 3-5 doses (week 18 or week 10). No case was detected after the fifth dose. ARIA-E occurred at doses of 6 and 10 mg/kg after 2 doses (week 6) and at week 30. Imaging findings generally resolved in 4-12 weeks, indicating that ARIA-E was reversible.

All subjects with ARIA-H events also had ARIA-E events. The incidence of ARIA-E was greater than the incidence of ARIA-H in each of the 3 mg/kg and 10 mg/kg treatment groups. The incidence of each event in the group receiving the 1 mg/kg doses was the same.

### C. Pre-Specified Interim Analysis #2

Pre-specified Interim Analysis #2 provided 54 week data for the 1, 3, and 10 mg/kg groups and the placebo group, as well as 26 week data for the 6 mg/kg group.

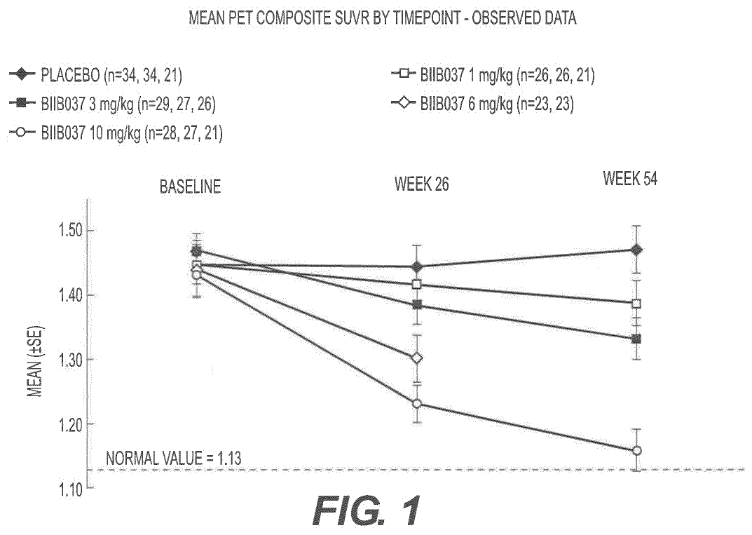

FIG. 1 shows the mean PET composite standardized uptake ratio values (SUVR) by time point based on observed data for each of the treatment groups. FIG. 1 shows that there was a reduction in amyloid burden in each of the treatment groups receiving antibody BIIB037 from baseline to week 26. There was a further reduction in amyloid burden in each of the treatment groups receiving BIIB037 between week 26 and week 54. The placebo group did not exhibit a corresponding reduction in amyloid burden.

FIG. 1 also shows that the reduction of amyloid burden by administration of BIIB037 was dose-dependent. Higher doses of BIIB037 were accompanied by a greater amyloid reduction in the brain using the amyloid scan. A similar effect was not observed in the placebo group.

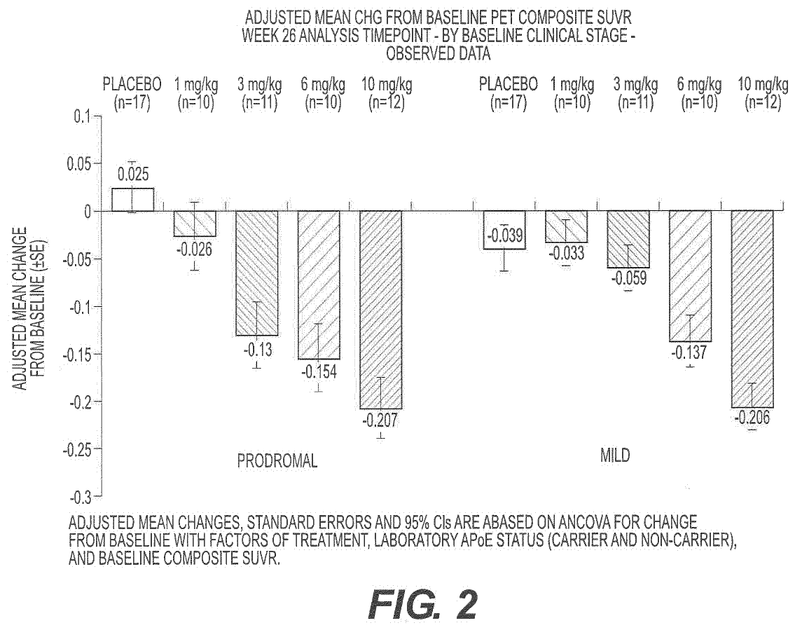

FIG. 2 shows the adjusted mean change from baseline PET composite SUVR at week 26 by baseline clinical stage, namely, prodromal or mild AD. FIG. 2 is based on observed data. FIG. 2 shows that amyloid reduction was dose-dependent in the amyloid scans.

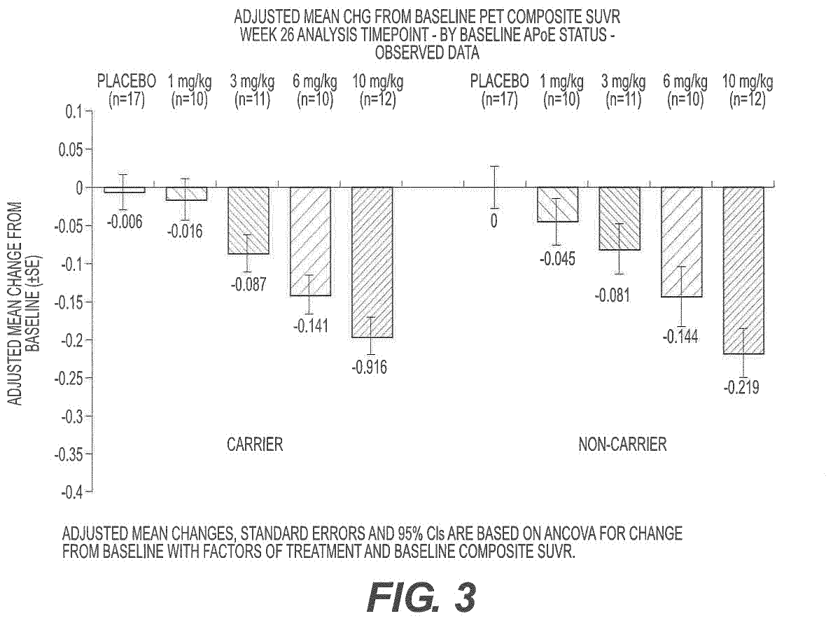

FIG. 3 shows the reduction in amyloid burden by ApoE4 status of the subjects. Both the carrier group and the non-carrier group showed a reduction in amyloid burden compared to the placebo. The reduction was dose-dependent in each case.

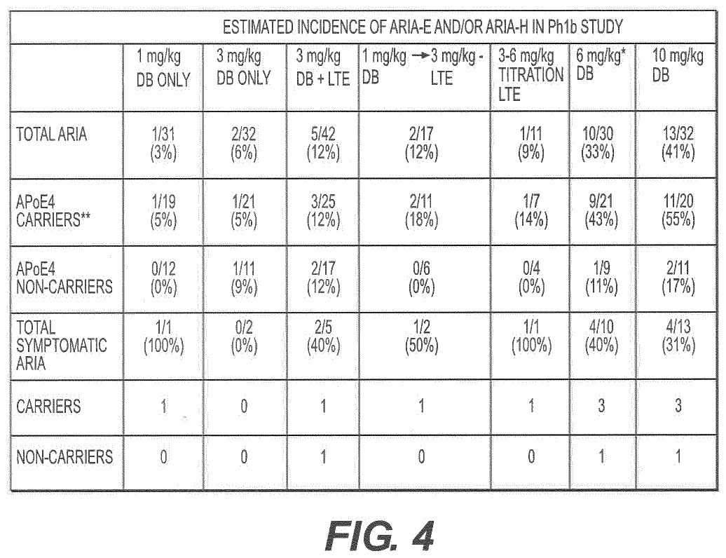

The incidence of ARIA-E and/or ARIA-H in the study was estimated. The results are shown in FIG. 4. The incidence of ARIA in ApoE4 carriers and ApoE4 non-carriers are also reported in FIG. 4. The incidence was dose-dependent and the ApoE4 carriage dependent at 6 and 10 mg/kg. The onset of ARIA-E was usually early in the course of treatment. ARIA-E was, in general, reversible. ARIA-H was stable. Imaging findings generally resolved in 4-12 weeks.

### D. Clinical Assessment of Patient Cognition

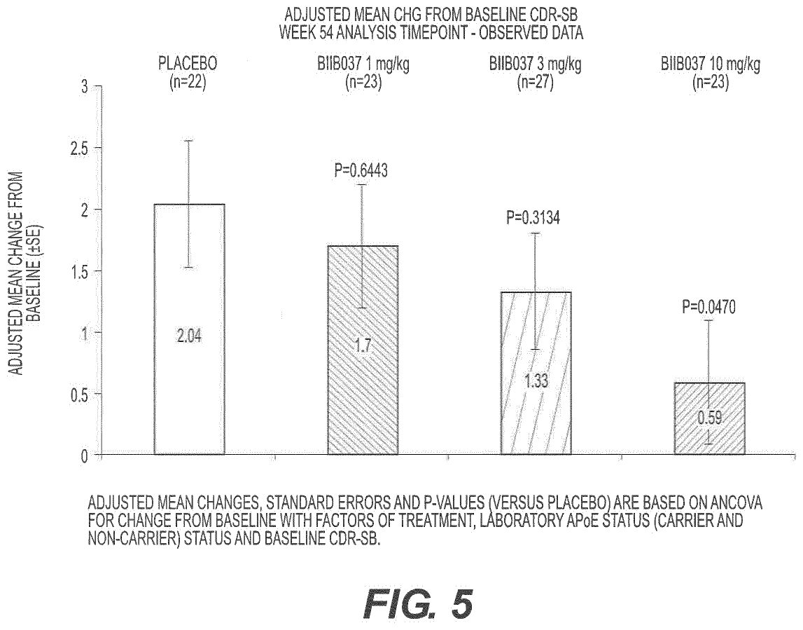

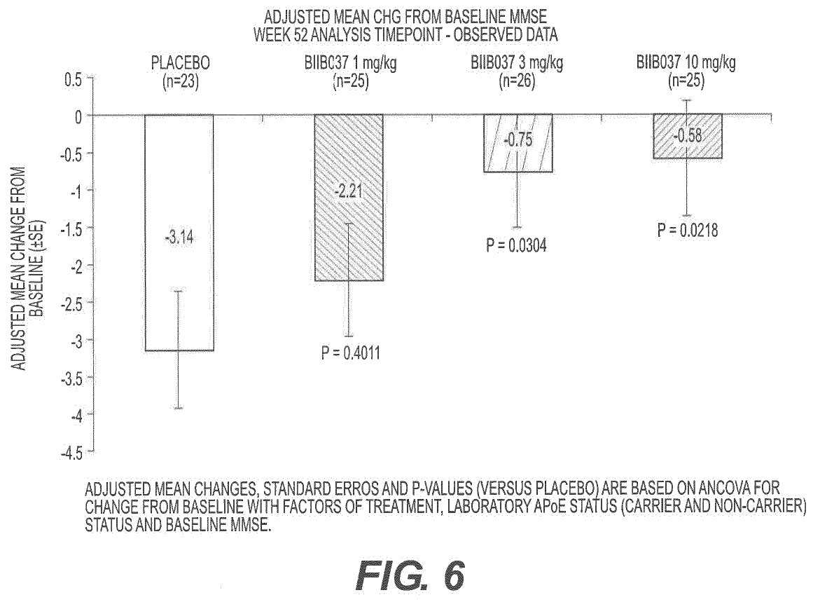

Clinical assessments were employed as indicators of changes in the symptoms of Alzheimer's disease in the patients treated. Specifically, changes from baseline were determined on the Clinical Dementia Rating (CDR) Scale and the Mini Mental State Examination (MMSE). The results of these assessments based on observed data are summarized in FIGS. 5 and 6.

FIG. 5 shows the adjusted mean change from baseline CDR-SB for patients receiving a placebo compared with patient populations receiving 1 mg/kg, 3 mg/kg, or 10 mg/kg of antibody BIIB037. Measurements were made at week 54 of treatment with the specified doses.

FIG. 6 shows the adjusted mean change from baseline MMSE for patients receiving a placebo compared with patient populations receiving 1 mg/kg, 3 mg/kg, or 10 mg/kg of antibody BIIB037. Measurements were made at week 54 of treatment with the specified doses.

### Example 7: Randomized, Double-Blind, Placebo-Controlled, Phase 1b Study of Aducanumab (BIIB037), an Anti-Aβ Monoclonal Antibody, in Patients with Prodromal or Mild Alzheimer's Disease: Interim Results by Disease Stage and ApoE ε4 Status

Aducanumab (BIIB037) is a human monoclonal antibody selective for aggregated forms of beta-amyloid (Aβ) peptide, including soluble oligomers and insoluble fibrils. A single ascending dose study of aducanumab demonstrated acceptable safety and in patients with mild-to-moderate AD at does up to 30 mg/kg. This Phase 1b study evaluated the safety, tolerability, pharmacokinetics (PK), and pharmacodynamics of aducanumab in patients with prodromal or mild AD.

The objective was to present interim safety and Aβ removal (change in florbetapir [18-AV-45] positron emission tomography [PET] results) with aducanumab by disease stage and ApoE ε4 status.

### Study Design

PRIME is a multicenter, randomized, double-blind, placebo-controlled, multiple-dose study [NCT01677572].

Patients were aged 50-90 years, had stable concomitant medications, had a Mini-Mental State Examination (MMSE) score ≥20 and met clinical and radiologic criteria as follows:
Prodromal AD: MMSE 24-30 spontaneous memory complaint; total free recall score ≤27 of the Free and Cued Selective Reminding Test; a global Clinical Dementia Rating (CDR) score of 0.5; absence of significant levels of impairment in other cognitive domains; essentially preserved activities of daily living and absence of dementia; had a positive florbetapir PET scan by visual assessment.
Mild AD: MMSE 20-26; global CDR 0.5 or 1.0; meeting National Institute on Aging and Alzheimer's Association core clinical criteria for probable AD; had a positive florbetapir PET scan by visual assessment.

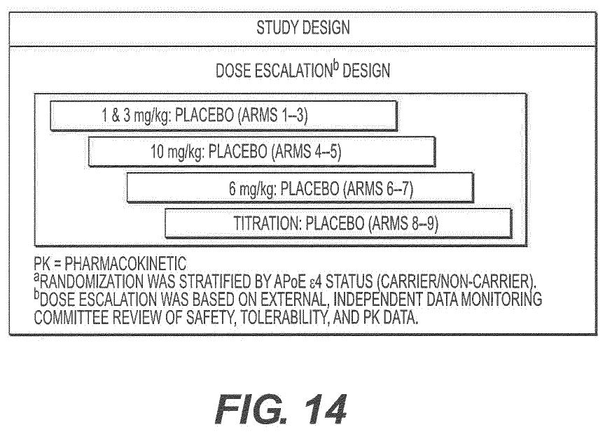

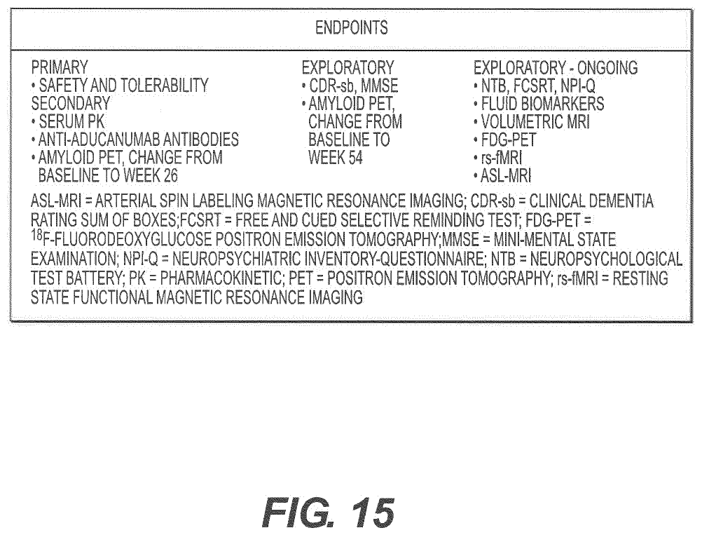

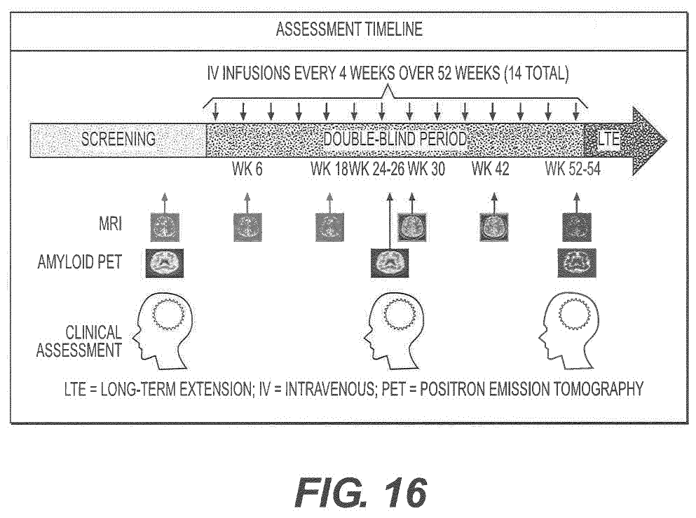

The PRIME study design is shown in FIG. 14. Patients (planned N=188) were randomized to 1 of 9 treatment arms (target enrollment: n=30 per active treatment arm) in a staggered, ascending dose design at a ratio of 3:1 active vs. placebo. Primary and secondary endpoints are presented in FIG. 15. The PRIME assessment timeline is shown in FIG. 16. PRIME is ongoing. For interim analysis, data were analyzed to Week 54 for the 1, 3, and 10 mg/kg arms and to Week 30 for the 6 mg/kg arm.

### Patients

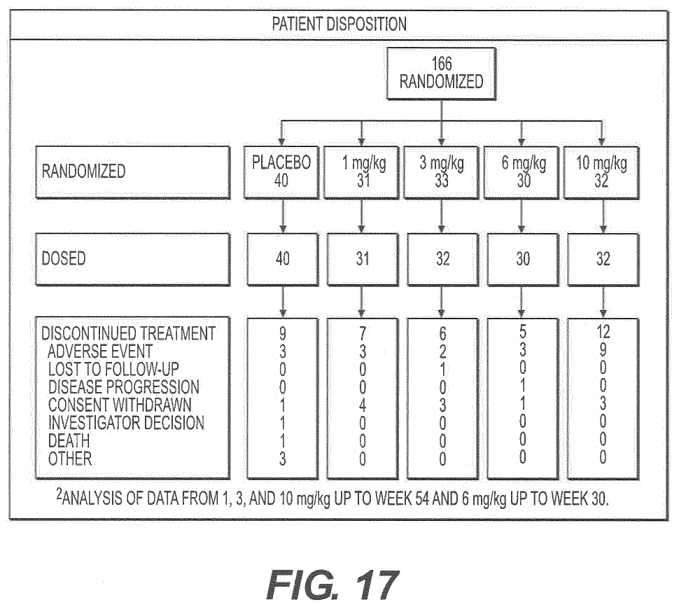

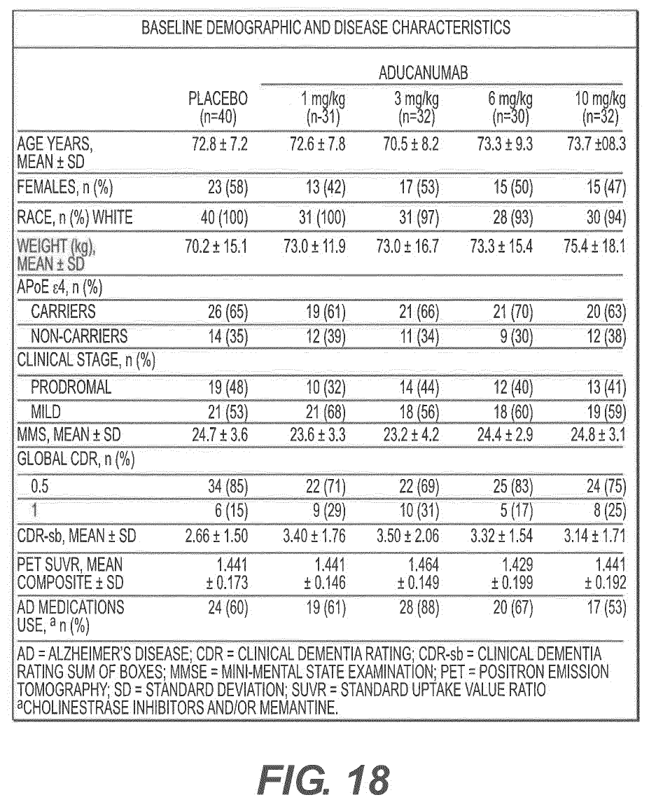

Of the 166 patients randomized, 165 were dosed; 107 (65%) were ApoE ε4 carriers, and 68 (41%) had prodromal AD. Patient disposition is shown in FIG. 17. Baseline demographic and disease characteristics were generally well balanced across treatment groups as shown in FIG. 18.

### Safety

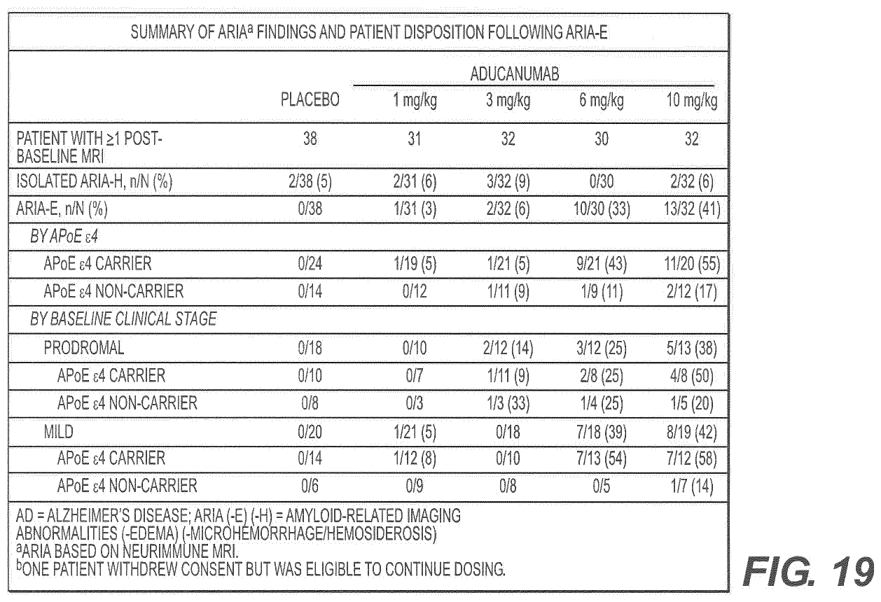

Adverse events (AE) were reported in 84%-98% of patients across treatment groups. The most common AE and serious AE (SAE) were amyloid-related imaging abnormalities (ARIA; based on MRI) (Table 9); other AEs/SAEs were consistent with the patient population. FIG. 19 provides a summary of ARIA findings and patient disposition following ARIA-E.

Three deaths were reported (2 with placebo, 1 with aducanumab 10 mg/kg); none were considered treatment related (2 occurred after study discontinuation).

Incidence of isolated ARIA-edema (ARIA-E) was dose- and ApoE ε4-status-dependent (FIG. 19):
Overall incidence of ARIA-E among ApoE ε4 carriers was 5%, 5%, 43%, and 55% for 1, 3, 6, and 10 mg/kg aducanumab, respectively, versus 0% for placebo.
Corresponding incidence among ApoE ε4 non-carriers was 0%, 9%, 11%, and 17% versus 0%.
Incidence of isolated ARIA-microhemorrhage/hemosiderosis (ARIA-H) was similar across doses and ApoE ε4 status (data not shown).

Based on small sample sizes, there was no apparent difference in incidence of ARIA-E between subjects with prodromal or mild AD when accounting for ApoE ε4 status (FIG. 19).

Most (92%) ARIA-E events were observed within the first 5 doses; 65% of ARIA-E events were asymptomatic.
When present, symptoms typically resolved within 4 weeks.
MRI findings typically solved within 4-12 weeks.

The majority of patients (54%) who developed ARIA-E continued treatment (93% of those who continued did so at a reduced dose); no patients developed recurrent ARIA-E. Treatment discontinuations in patients with ARIA-E were consistent across mild and prodromal subgroups (data not shown).

There were no significant changes in chemistry, hematology, urinalysis, electro-cardiogram, or vital signs.

### Brain Aβ Plaque Reduction

Brain Aβ plaque reduction was evaluated by composite SUVR from a volume of 6 regions; frontal, parietal, lateral temporal, sensorimotor, anterior cingulate, and posterior cingulate.

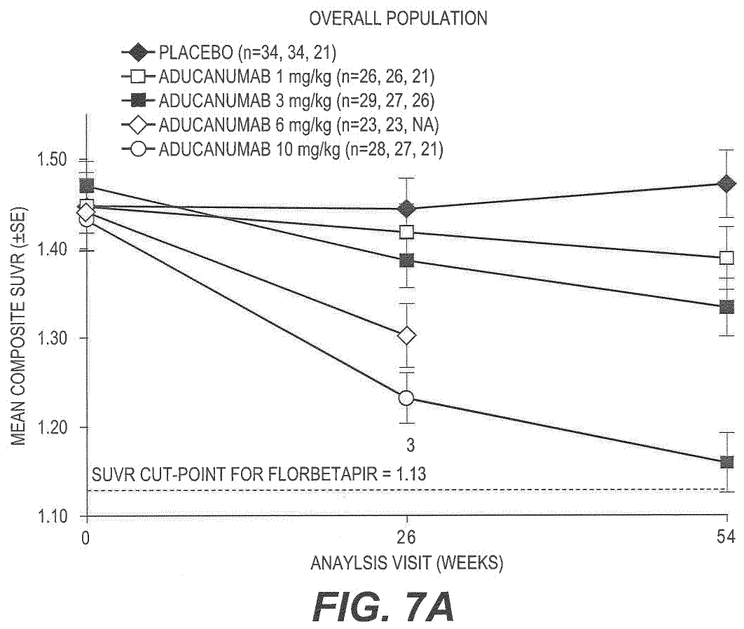

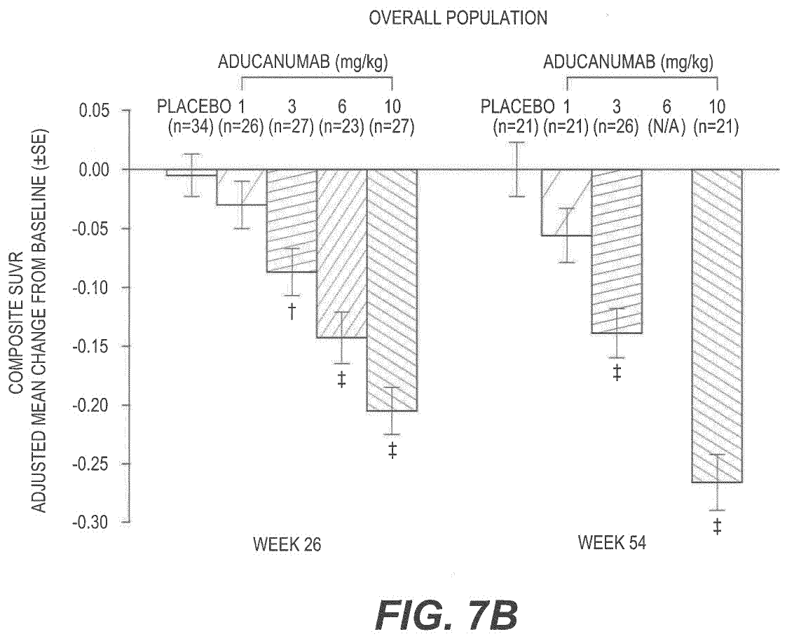

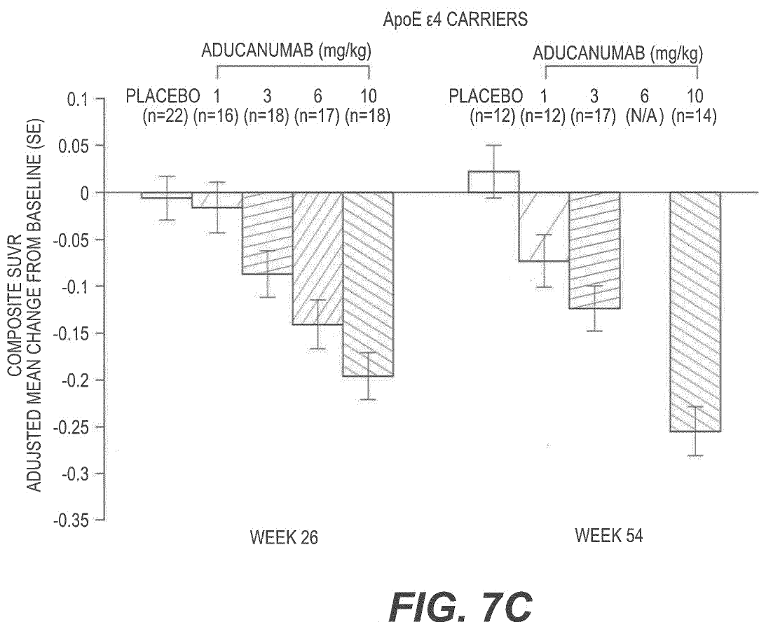

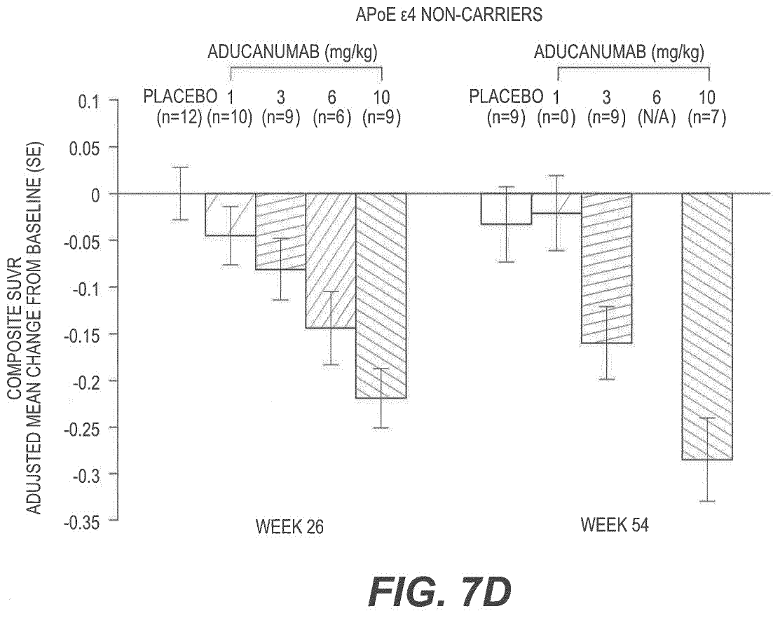

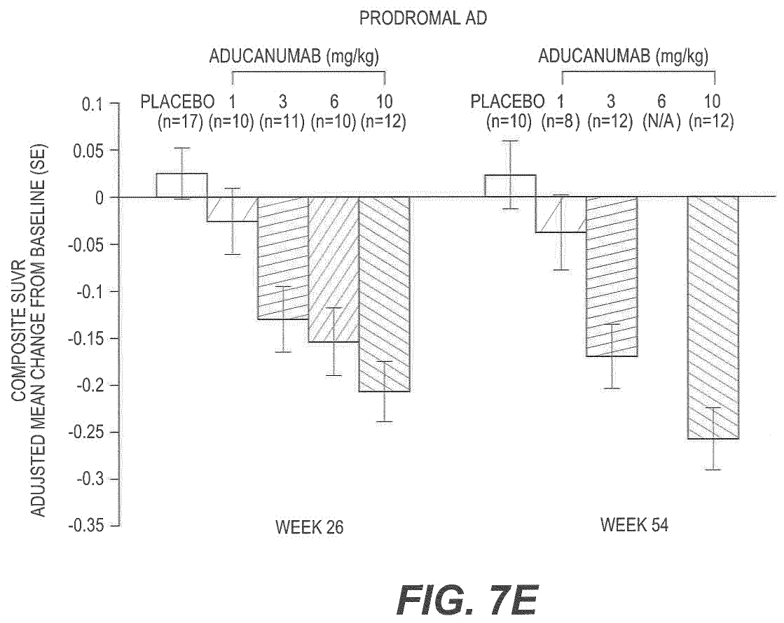

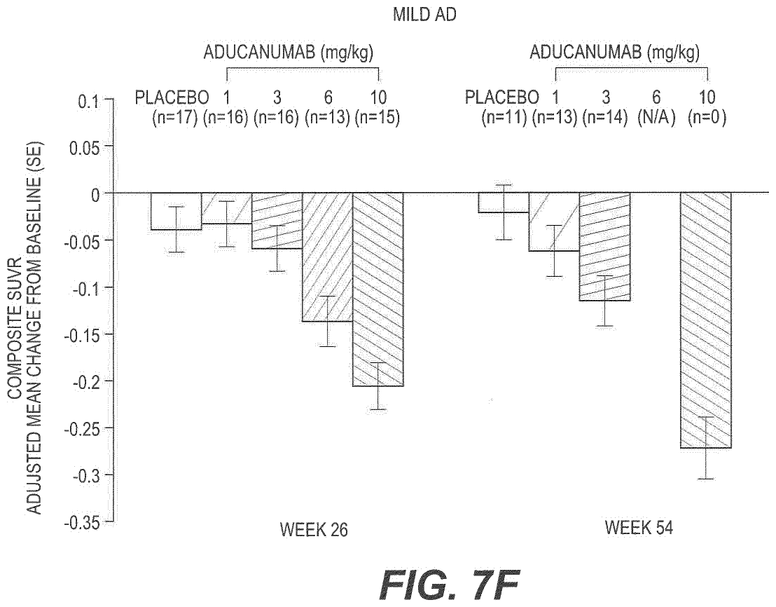

Dose- and time-dependent reductions in brain Aβ plaque (evidenced by SUVR reduction) at weeks 26 and 54 were generally consistent across mild and prodromal AD subgroups and across ApoE ε4 carriers and non-carriers within the doses tested as shown in FIG. 7.

### Clinical Endpoints

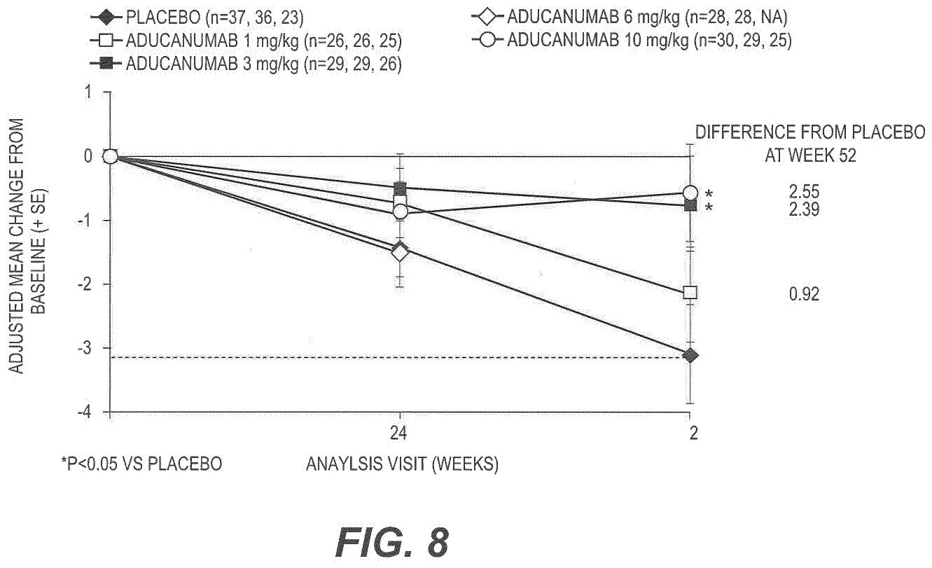

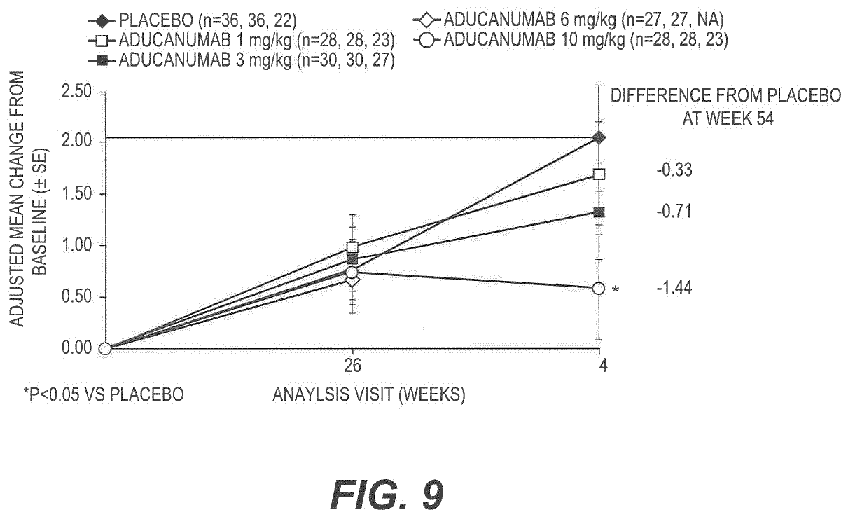

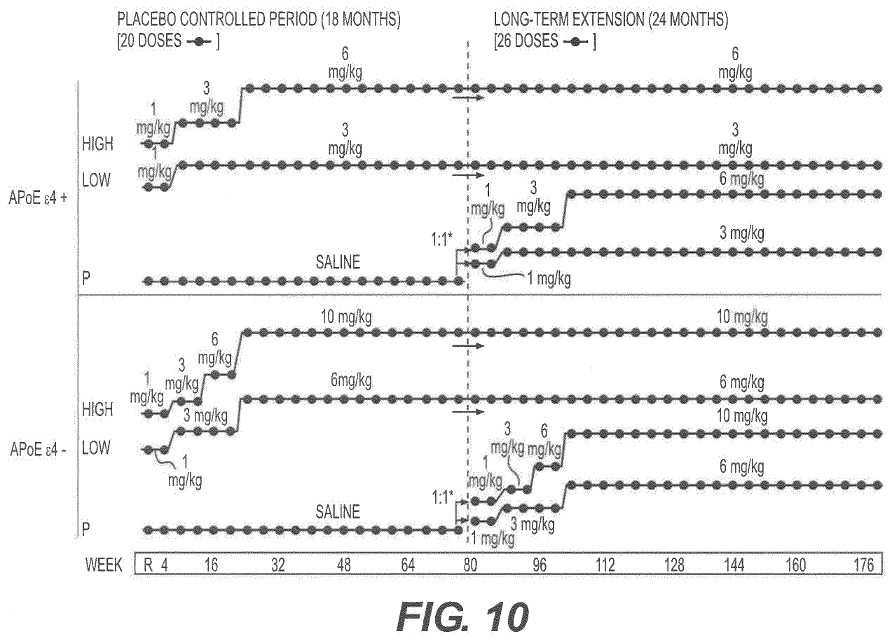

There was statistically significant dose-dependent slowing of decline on the exploratory endpoints, MMSE (FIG. 8) and CDR-sb (FIG. 9) at 1 year.

### CONCLUSIONS

There was a significant dose- and time-dependent reduction of brain Aβ plaques as measured by PET Imaging versus placebo. This effect was evident at 6 months and 1 year of treatment.

The pattern of the aducanumab effect versus placebo on Aβ plaque reduction was generally consistent across disease stage and ApoE ε4 status.

A statistically significant dose-dependent slowing of decline on MMSE and CDR-sb was observed at 1 year.

Aducanumab demonstrated an acceptable safety profile over 54 weeks. ARIA was the main safety and tolerability finding and was able to be monitored and managed. The incidence of ARIA was dose- and ApoE-ε4-status-dependent. ARIA was usually observed early in the course of treatment and was asymptomatic or with mild, transient symptoms.

### Interim Analysis #3

Interim Analysis #3 includes data to 54 weeks for the 6 mg/kg arm and the corresponding placebo arm (which is incorporated into the pooled placebo population for the analysis).

Brain Aβ Plaque Reduction

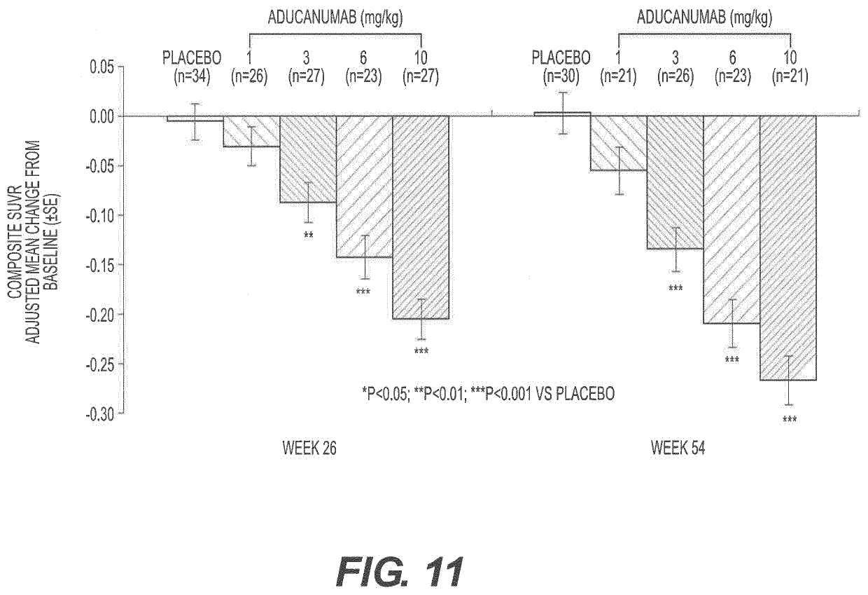

Brain Aβ plaque reduction was evaluated by composite SUVR from a volume of 6 regions; frontal, parietal, lateral temporal, sensorimotor, anterior cingulate, and posterior cingulate. As shown in FIG. 11, there was a dose-dependent reduction in brain Aβ plaque (evidenced by SUVR reduction) at week 54.

### Clinical Endpoints

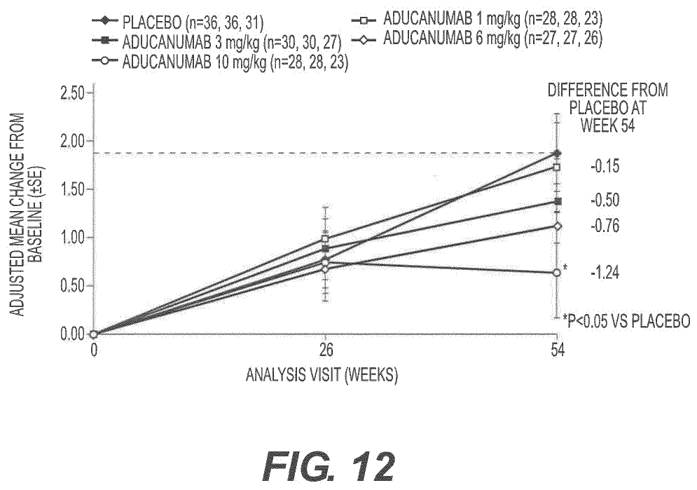

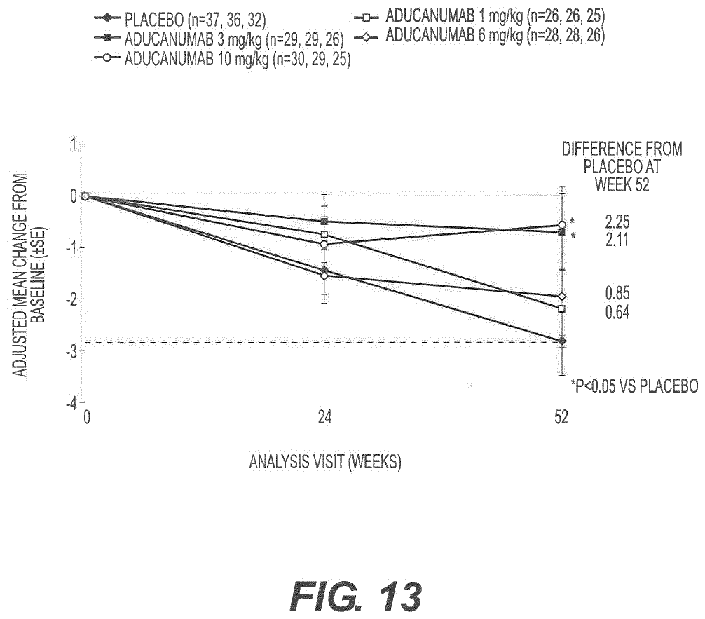

There was statistically significant dose-dependent slowing of decline on the exploratory endpoints, MMSE (FIG. 13) and CDR-sb (FIG. 12) at 1 year.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating Alzheimer's disease (AD) in a human subject in need thereof, the method comprising:
administering to the human subject multiple doses of an anti-beta-amyloid antibody, wherein the subject develops an Amyloid Related Imaging Abnormality (ARIA) during treatment with the anti-beta-amyloid antibody, wherein the ARIA is (i) ARIA-E that is moderate or severe and is accompanied by no clinical symptoms, (ii) ARIA-E that is mild, moderate, or severe and is accompanied by mild, moderate, severe, or serious clinical symptoms, (iii) ARIA-H with 5 to 9 cumulative microhemorrhages and accompanied by no clinical symptoms, (iv) ARIA-H with 1 to 9 cumulative microhemorrhages and accompanied by mild, moderate, severe, or serious clinical symptoms, (v) ARIA-H with 2 cumulative areas of superficial siderosis and accompanied by no clinical symptoms, or (vi) ARIA-H with 1 or 2 cumulative areas of superficial siderosis and accompanied by mild, moderate, severe, or serious clinical symptoms;
suspending administration to the subject of the anti-beta-amyloid antibody, after onset of the ARIA, until the ARIA resolves; and
resuming administration to the subject of the same dose of the anti-beta-amyloid antibody that was administered immediately prior to the subject developing the ARIA,
wherein the anti-beta-amyloid antibody comprises a heavy chain variable region (VH) and a light chain variable region (VL),
wherein the VH comprises a first complementarity determining region (VHCDR1) with the amino acid sequence of SEQ ID NO:3, a VHCDR2 with the amino acid sequence of SEQ ID NO:4, and a VHCDR3 with the amino acid sequence of SEQ ID NO:5, and
wherein the VL comprises a VLCDR1 with the amino acid sequence of SEQ ID NO:6, a VLCDR2 with the amino acid sequence of SEQ ID NO:7, and a VLCDR3 with the amino acid sequence of SEQ ID NO:8.

2. The method of claim 1, wherein the multiple doses of the anti-beta-amyloid antibody are doses of the same amount.

3. The method of claim 1, wherein the multiple doses of the anti-beta-amyloid antibody comprise doses of different amounts.

4. The method of claim 2, wherein the multiple doses are 1 mg/kg of body weight of the subject.

5. The method of claim 2, wherein the multiple doses are 3 mg/kg of body weight of the subject.

6. The method of claim 2, wherein the multiple doses are 6 mg/kg of body weight of the subject.

7. The method of claim 2, wherein the multiple doses are 10 mg/kg of body weight of the subject.

8. The method of claim 2, wherein the multiple doses are 15 mg/kg of body weight of the subject.

9. The method of claim 2, wherein the multiple doses are 30 mg/kg of body weight of the subject.

10. The method of claim 3, wherein the multiple doses comprise 1 mg/kg and 3 mg/kg of body weight of the subject.

11. The method of claim 3, wherein the multiple doses comprise 1 mg/kg, 3 mg/kg, and 6 mg/kg of body weight of the subject.

12. The method of claim 3, wherein the multiple doses comprise 1 mg/kg, 3 mg/kg, 6 mg/kg, and 10 mg/kg of body weight of the subject.

13. The method of claim 3, wherein the subject is an ApoE4 carrier and the multiple doses comprise two or more of the doses 1 mg/kg, 3 mg/kg, or 6 mg/kg of body weight of the subject.

14. The method of claim 3, wherein the subject is an ApoE4 non-carrier and the multiple doses comprise two or more of the doses 1 mg/kg, 3 mg/kg, 6 mg/kg, 10 mg/kg, 15 mg/kg, or 30 mg/kg of body weight of the subject.

15. The method of any one of claims 1, 3, or 10 to 14, further comprising subsequently administering the anti-beta-amyloid antibody at a dose that is higher than the dose that is administered upon resumption of administration after resolution of the ARIA.

16. The method of any one of claims 1 to 15, wherein the multiple doses are administered at intervals of 4 weeks.

17. The method of any one of claims 1 to 16, wherein the number of multiple doses administered to the subject prior to the onset of the ARIA is 2 to 14.

18. The method of any one of claims 1 to 14, wherein the number of multiple doses administered to the subject prior to the onset of the ARIA is 2 to 5.

19. The method of claim 1, wherein administering to the human subject multiple doses of the anti-beta-amyloid antibody comprises, in order, beginning with step (a) performing two or more of the following administering steps prior to the onset of the ARIA:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 3 mg/kg of body weight of the subject;
(f) 4 weeks after step (e), administering the antibody to the subject in an amount of 3 mg/kg of body weight of the subject;
(g) 4 weeks after step (f), administering the antibody to the subject in an amount of 6 mg/kg of body weight of the subject; and
(h) in consecutive intervals of 4 weeks after step (g), administering the antibody to the subject in an amount of 6 mg/kg of body weight of the subject.

20. The method of claim 19, wherein the method comprises, after resolution of the ARIA, performing from the following administering steps, in order, those steps that were not performed prior to the onset of the ARIA:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 3 mg/kg of body weight of the subject;
(f) 4 weeks after step (e), administering the antibody to the subject in an amount of 3 mg/kg of body weight of the subject;
(g) 4 weeks after step (f), administering the antibody to the subject in an amount of 6 mg/kg of body weight of the subject; and
(h) in consecutive intervals of 4 weeks after step (g), administering the antibody to the subject in an amount of 6 mg/kg of body weight of the subject.

21. The method of claim 1, wherein administering to the human subject multiple doses of the anti-beta-amyloid antibody comprises, in order, beginning with step (a) performing two or more of the following administering steps prior to the onset of the ARIA:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 6 mg/kg of body weight of the subject;
(f) 4 weeks after step (e), administering the antibody to the subject in an amount of 6 mg/kg of body weight of the subject; and
(g) in consecutive intervals of 4 weeks after step (f), administering the antibody to the subject in an amount of 10 mg/kg of body weight of the subject, wherein the subject is an ApoE4 non-carrier.

22. The method of claim 21, wherein the method comprises, after resolution of the ARIA, performing from the following administering steps, in order, those steps that were not performed prior to the onset of the ARIA:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 6 mg/kg of body weight of the subject;
(f) 4 weeks after step (e), administering the antibody to the subject in an amount of 6 mg/kg of body weight of the subject; and
(g) in consecutive intervals of 4 weeks after step (f), administering the antibody to the subject in an amount of 10 mg/kg of body weight of the subject.

23. The method of claim 1, wherein administering to the human subject multiple doses of the anti-beta-amyloid antibody comprises:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject; and
(c) in consecutive intervals of 4 weeks after step (b), administering the antibody to the subject in an amount of 3 mg/kg of body weight of the subject,
wherein the subject is an ApoE4 carrier.

24. The method of any one of claims 1 to 23, wherein, after resuming administration of the anti-beta-amyloid antibody, the human subject develops a second ARIA that is (i) ARIA-E that is moderate or severe and is accompanied by no clinical symptoms, (ii) ARIA-E that is mild, moderate, or severe and is accompanied by mild, moderate, severe, or serious clinical symptoms, (iii) ARIA-H with 5 to 9 cumulative microhemorrhages and accompanied by no clinical symptoms, (iv) ARIA-H with 1 to 9 cumulative microhemorrhages and accompanied by mild, moderate, severe, or serious clinical symptoms, (v) ARIA-H with 2 cumulative areas of superficial siderosis and accompanied by no clinical symptoms, or (vi) ARIA-H with 1 or 2 cumulative areas of superficial siderosis and accompanied by mild, moderate, severe, or serious clinical symptoms, and the method further comprises:
suspending administration of the anti-beta-amyloid antibody to the subject until the second ARIA resolves; and
resuming administration of the anti-beta-amyloid antibody to the subject at a dose that is lower than the dose that was administered to the subject immediately prior to the subject developing the second ARIA.

25. The method of any one of claims 1 to 24, wherein the ARIA is accompanied by no clinical symptoms.

26. The method of any one of claims 1 to 24, wherein the ARIA is accompanied by mild clinical symptoms.

27. The method of any one of claims 1 to 24, wherein the ARIA is accompanied by moderate clinical symptoms.

28. The method of any one of claims 1 to 24, wherein the ARIA is accompanied by severe clinical symptoms.

29. The method of any one of claims 1 to 28, wherein administration is performed intravenously.

30. The method of any one of claims 1 to 29, wherein:
the VH consists of SEQ ID NO:1; and
the VL consists of SEQ ID NO:2.

31. The method of any one of claims 1 to 30, wherein the antibody comprises a human IgG1 constant region.

32. The method of any one of claims 1 to 29, wherein the antibody comprises a heavy chain and a light chain, wherein:
the heavy chain consists of SEQ ID NO:10; and
the light chain consists of SEQ ID NO:11.

33. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 3 mg/kg of body weight of the subject;
(f) 4 weeks after step (e), administering the antibody to the subject in an amount of 3 mg/kg of body weight of the subject;
(g) 4 weeks after step (f), administering the antibody to the subject in an amount of 6 mg/kg of body weight of the subject;
(h) 4 weeks after step (g), administering the antibody to the subject in an amount of 6 mg/kg of body weight of the subject;
(i) 4 weeks after step (h), administering the antibody to the subject in an amount of 6 mg/kg of body weight of the subject;
(j) 4 weeks after step (i), administering the antibody to the subject in an amount of 6 mg/kg of body weight of the subject;
(k) 4 weeks after step (j), administering the antibody to the subject in an amount of 6 mg/kg of body weight of the subject; and
(l) in consecutive intervals of 4 weeks after step (k), administering the antibody to the subject in an amount of 10 mg/kg of body weight of the subject.

34. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 3 mg/kg of body weight of the subject;
(b) 4 weeks after step (a), administering the antibody to the subject in an amount of 3 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 6 mg/kg of body weight of the subject;
(f) 4 weeks after step (e), administering the antibody to the subject in an amount of 6 mg/kg of body weight of the subject;
(g) 4 weeks after step (f), administering the antibody to the subject in an amount of 6 mg/kg of body weight of the subject;
(h) 4 weeks after step (g), administering the antibody to the subject in an amount of 6 mg/kg of body weight of the subject;
(i) 4 weeks after step (h), administering the antibody to the subject in an amount of 6 mg/kg of body weight of the subject; and
(j) in consecutive intervals of 4 weeks after step (i), administering the antibody to the subject in an amount of 10 mg/kg of body weight of the subject.

35. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 1 mg/kg of body weight of the subject;
(b) 4 weeks after step (a), administering the antibody to the subject in an amount of 1 mg/kg of body weight of the subject;
(c) 4 weeks after step (b), administering the antibody to the subject in an amount of 3 mg/kg of body weight of the subject;
(d) 4 weeks after step (c), administering the antibody to the subject in an amount of 3 mg/kg of body weight of the subject;
(e) 4 weeks after step (d), administering the antibody to the subject in an amount of 6 mg/kg of body weight of the subject;
(f) 4 weeks after step (e), administering the antibody to the subject in an amount of 6 mg/kg of body weight of the subject; and
(g) in consecutive intervals of 4 weeks after step (f), administering the antibody to the subject in an amount of 10 mg/kg of body weight of the subject.

36. A method for treating Alzheimer's disease in a human subject in need thereof, the method comprising administering to the human subject multiple doses of an anti-beta-amyloid antibody, wherein the multiple doses are administered as follows:
(a) administering the anti-beta-amyloid antibody to the subject in an amount of 3 mg/kg of body weight of the subject;
(b) 4 weeks after step (a), administering the antibody to the subject in an amount of 3 mg/kg of body weight of the subject;
(c) 4 weeks after step (b), administering the antibody to the subject in an amount of 6 mg/kg of body weight of the subject;
(d) 4 weeks after step (c), administering the antibody to the subject in an amount of 6 mg/kg of body weight of the subject; and
(e) in consecutive intervals of 4 weeks after step (d), administering the antibody to the subject in an amount of 10 mg/kg of body weight of the subject.

37. The method of any one of claims 33 to 36, wherein the human subject is an ApoE4 carrier.

38. The method of any one of claims 1 to 37, wherein administration is performed intravenously.

39. The method of any one of claims 1 to 38, wherein the anti-beta-amyloid antibody comprises a heavy chain variable region (VH) and a light chain variable region (VL), wherein:
the VH consists of SEQ ID NO:1; and
the VL consists of SEQ ID NO:2.

40. The method of any one of claims 1 to 39, wherein the antibody comprises a human IgG1 constant region.

41. The method of any one of claims 1 to 38, wherein the anti-beta-amyloid antibody comprises a heavy chain and a light chain, wherein:
the heavy chain consists of SEQ ID NO:10; and
the light chain consists of SEQ ID NO:11.
